(19) United States
(12) Patent Application Publication
(10) Pub. No.: US 2025/0347697 A1
(43) Pub. Date: Nov. 13, 2025

(54) Accurate Method for Generating a Phase Diagram of a Polymer

(71) Applicant: MAX-PLANCK-GESELLSCHAFT ZUR FÖRDERUNG DER WISSENSCHAFTEN E.V., Munich (DE)

(72) Inventors: Anatol W. Fritsch, Dresden (DE); Juan Manuel Iglesias Artola, Dresden (DE); Anthony A. Hyman, Dresden (DE)

(21) Appl. No.: 18/871,218

(22) PCT Filed: Jun. 5, 2023
(86) PCT No.: PCT/EP2023/064947

§ 371 Date: Dec. 3, 2024

(30) Foreign Application Priority Data
Jun. 3, 2022 (EP) 22177321.1

(51) Int. Cl. G01N 33/68; G01N 33/569

(52) U.S. Cl. CPC G01N 33/6842; G01N 33/56966; G01N 33/56983; G01N 2333/01; G01N 2333/43534; G01N 2458/15

(57) ABSTRACT

The present invention refers to a method for generating a binodal curve of a polymer in a system by determining accurate values of both concentration of dilute and condensed phases of a polymer, in particular, protein or polynucleic acid, under different condition, such as at different temperatures and salt concentrations. Furthermore, the present invention refers to an assay method for identify bioactive compound(s), comprising the inventive method for generating a binodal curve of a polymer in a system.

The present invention refers to a method for generating a binodal curve of a polymer in a system by determining accurate concentration values for both the dilute and condensed phases of a polymer, in particular, protein or polynucleic acid, under different conditions, such as at different temperatures, pH, salt concentrations, pressure, crowding agents, buffer compositions or in a mixture of polymers. The method can be applied to in vitro and in vivo systems alike.

Furthermore, the present invention refers to an assay method for identifying bioactive compound(s), comprising the inventive method for generating a binodal curve of a polymer in a system.

### BACKGROUND OF THE INVENTION

Protein phase separation has become a widely studied phenomenon in biology with implications in cell metabolism and disease. An accurate study of phase separating proteins relies on the precise determination of the binodal curves. The binodal curves in these phase diagrams give information on the protein concentration required for condensate formation and the respective concentration inside the condensate at defined external conditions (temperature, salt, pH, pressure, buffer composition, crowding agent, or in a mixture of polymers). However, it has proven difficult to measure accurate binodal curves and, in general, phase diagrams.

Protein condensates are present in a wide variety of cells where they perform fundamental functions in development (e. g. P granules in C. elegans), ageing (e.g. stress granules) and in diverse cellular processes (e.g. transcription). They are known to form through a thermodynamic process, referred to as phase separation that results from favourable interactions between proteins and between proteins and polynucleic acids. The strength and nature of these intermolecular interactions are governed by the protein sequence and secondary structure, and by the cellular environment. Changes in sequence and environment can therefore dictate the protein concentrations necessary to achieve phase separation and the resulting material properties of the condensates. Temperature, salt concentration and pH are important external control parameters of thermodynamic phase separation.

Typically, high salt concentrations lead to condensate dissolution by shielding charge interactions between and within proteins. A similar effect is observed with temperature, where the solvation state and the secondary structure of the protein are affected. From these considerations, it follows that to understand the phase separation of a given protein, its phase diagrams must be constructed in respect to the relevant variables under study. The complete phase diagram maps the control parameter(s) (temperature, salt concentrations, etc.) with the dilute and the condensed phases of a system via a binodal curve.

Thus far it has proven challenging to produce accurate phase diagrams for a wide variety of proteins as a consequence of sample constrains and technical requirements. Ideally, it should be possible to determine binodal curves using a small volume of protein sample and readily accessible laboratory equipment. While dilute phase protein concentrations, or saturation concentrations, have been reported in numerous studies; the condensed phase concentration is more complicated to be measured and usually requires large amounts of protein (Brady, J. P. et al. Structural and hydrodynamic properties of an intrinsically disordered region of a germ cell-specific protein on phase separation. PNAS, 2017, 114, E8194-E8203; McCall, P. M. et al. Quantitative phase microscopy enables precise and efficient determination of biomolecular condensate composition. 2020.10.25.352823; Bracha, D. et al. Mapping Local and Global Liquid Phase Behavior in Living Cells Using Photo-Oligomerizable Seeds. Cell, 2018, 175, p. 1467-1480.).

US 2021/350 875 A1 discloses a high-throughput method and system for mapping intracellular phase diagrams. To this extent, a plurality of cells each cell expressing a phase separation or aggregation system capable of being controlled by at least one wavelength of light the phase separation or aggregation system comprising a target protein or a target protein and a fluorescent protein or attached fluorophore are placed in a well. A biological agent is added to the well at the first concentration and the well is irradiated with a wavelength of light, in a constant or pulsed fashion allowing the phase separation or aggregation system to form condensates. The cells are irradiated with an additional wavelength of light to cause the fluorescent protein or an attached fluorophore to fluoresce. Then a quantification of phase separation or aggregation based on an amount of fluorescence within a first region and a second region of the plurality of cells, the first region containing a condensate and the second region not containing a condensate is performed.

Bracha et al. (bioRxiv, 2018-03-16, p. 1-16 (XP055694847)) disclose a device for determining a binodal curve that measures the concentration of cores outside of the droplets named core dilute and protein concentration in droplets named core dense. The condensate volume and the total volume are also measured.

This is in part because of the difficulties that arise from quantitative fluorescence-based approaches as a consequence of fluorophore quenching caused by changes in the environment inside the condensate and optical limitations. Resourcing to bulk approaches is often not accessible due to the amount of protein that would be required for measurement.

In this work the inventors present a simple and accurate approach that is based on mass and volume conservation and defined reaction volumes to determine binodal curves for phase separating polymers.

It is the objective of the present invention to provide a method for determining accurate concentration values for both the dilute and condensed phases of a polymer, in particular, protein or polynucleic acid, under different conditions, such as at different temperatures, salt concentrations or in the presence of other polymers in a system for generating binodal curves for a phase diagram of said polymer.

The present invention can be applied to in vitro and in vivo systems alike.

Furthermore, the present invention refers to an assay method for identify bioactive compound(s), comprising the inventive method for generating a binodal curve of a polymer in a system.

In this invention, a simple and accurate method for producing binodal curves for phase diagrams is provided by determining both dilute and condensed phase concentrations of polymer mixtures. The method of the present invention overcomes the above-mentioned difficulties. It builds upon an accurate determination of the condensed phase volume fraction in an environment of known volume and a titration of polymer concentration. It allows determining accurate phase diagrams in a rapid manner.

The objective of the present invention is solved by the teaching of the independent claims. Further advantageous features, aspects and details of the invention are evident from the dependent claims, the description, the figures, and the examples of the present application.

### BRIEF SUMMARY OF THE INVENTION

The present invention refers to a method for determining a concentration (c_{con}) of a polymer in a condensed phase and a concentration (c_{dil}) of the polymer in a dilute phase in a system comprising:
A) determining a total volume (Vₜₒₜ) of the system and a total concentration (cₜₒₜ) of the polymer;
B) triggering phase separation of the polymer into a condensed phase and a dilute phase in said system;
C) determining a volume fraction (V_{con}/Vₜₒₜ) of a condensed phase of the polymer in said system;
D) determining a concentration (c_{con}) of the condensed phase of the polymer and a concentration (c_{dil}) of the dilute phase of the polymer in said system by using a following linear form equation:

$$\frac{{V}_{con}}{{V}_{tot}} = \frac{1}{{c}_{con} - {c}_{dil}} \cdot {c}_{tot} - \frac{{c}_{dil}}{{c}_{con} - {c}_{dil}} ,$$

E) changing at least one condition of said system and repeating the steps A) to D).

Preferably, the polymer is a protein, or a polynucleic acid, preferably RNA, DNA, or a mixture of a protein and RNA, or a mixture of a protein and DNA.

The inventors have derived a linear relationship between the volume fraction (V_{con}/Vₜₒₜ) of the condensed phase in a closed system and the total concentration of the polymer. Therefore, in a closed system, in a demixed state having two coexisting phases, a dilute and a condensed phase, the concentration of a polymer in both phases can be determined accurately from the volume fraction (V_{con}/Vₜₒₜ) of the polymer in the condensed phase at different total polymer concentrations cₜₒₜ. The condensed phase concentration is usually complicated to be measured and requires large amounts of protein when being directly measured (see Brady, J. P. et al. Structural and hydrodynamic properties of an intrinsically disordered region of a germ cell-specific protein on phase separation. PNAS, 2017, 114, E8194-E8203). With the inventive methods described herein, the polymer concentration in both phases can be determined accurately from easily obtainable parameters in a single experiment.

In other words, the present invention refers to a method for simultaneously determining a concentration (c_{con}) of a polymer in a condensed phase and a concentration (c_{dil}) of the polymer in a dilute phase in a system comprising:
A) determining a total volume (Vₜₒₜ) of the system and a total concentration (cₜₒₜ) of the polymer;
B) triggering phase separation of the polymer into a condensed phase and a dilute phase in said system;
C) determining a volume fraction (V_{con}/Vₜₒₜ) of a condensed phase of the polymer in said system;
D) determining a concentration (c_{con}) of the condensed phase of the polymer and a concentration (c_{dil}) of the dilute phase of the polymer in said system by using a following linear form equation:

$$\frac{{V}_{con}}{{V}_{tot}} = \frac{1}{{c}_{con} - {c}_{dil}} \cdot {c}_{tot} - \frac{{c}_{dil}}{{c}_{con} - {c}_{dil}} ,$$

E) changing at least one condition of said system and repeating the steps A) to D).

Reworded, the present invention refers to a method for determining a concentration (c_{con}) of a polymer in a condensed phase and a concentration (c_{dil}) of the polymer in a dilute phase in a system comprising:
A) providing specimens of a system comprising the polymer, each specimen having a total volume Vₜₒₜ and a different total concentration (cₜₒₜ) of the polymer;
B) triggering phase separation of the polymer into a condensed phase and a dilute phase in each specimen;
C) determining a volume fraction (V_{con}/Vₜₒₜ) of a condensed phase of the polymer in each specimen;
D) determining a concentration (c_{con}) of the condensed phase of the polymer and a concentration (c_{dil}) of the dilute phase of the polymer in each specimen by using a following linear form equation:

$$\frac{{V}_{con}}{{V}_{tot}} = \frac{1}{{c}_{con} - {c}_{dil}} \cdot {c}_{tot} - \frac{{c}_{dil}}{{c}_{con} - {c}_{dil}} ,$$

E) changing at least one condition of said system and repeating the steps A) to D).

Determining the polymer concentration in both phases in a single experiment also allows accurate binodal curve determination and thus phase diagram construction in a rapid manner. Conventional methods for binodal curve generation of a polymer are based on the determination of the concentration of the polymer in a dilute phase only and allow therefore only the determination of one side of the coexistence curve (binodal) in a single experiment. Thus, for the generation of a complete binodal curve and a complete phase diagram multiple sample preparations and experiments are required in order to obtain the concentration in the condensed phase.

In other words, the present invention refers to a method for determining a concentration (c_{con}) of a polymer in a condensed phase and a concentration (c_{dil}) of the polymer in a dilute phase in a system for generating a binodal curve comprising:
A) determining a total volume (Vₜₒₜ) of the system and a total concentration (cₜₒₜ) of the polymer;
B) triggering phase separation of the polymer into a condensed phase and a dilute phase in said system;
C) determining a volume fraction (V_{con}/Vₜₒₜ) of a condensed phase of the polymer in said system;
D) determining a concentration (c_{con}) of the condensed phase of the polymer and a concentration (c_{dil}) of the dilute phase of the polymer in said system by using a following linear form equation:

$$\frac{{V}_{c o n}}{{V}_{t o t}} = \frac{1}{{c}_{c o n} - {c}_{d i l}} \cdot {c}_{tot} - \frac{{c}_{d i l}}{{c}_{c o n} - {c}_{d i l}} ,$$

E) changing at least one condition of said system and repeating the steps A) to D).

Also, the present invention refers to a method for generating a binodal curve of a polymer in a system comprising:
A) determining a total volume (Vₜₒₜ) of the system and a total concentration (cₜₒₜ) of the polymer;
B) triggering phase separation of the polymer into a condensed phase and a dilute phase in said system;
C) determining a volume fraction (V_{con}/Vₜₒₜ) of a condensed phase of the polymer in said system;
D) determining a concentration (c_{con}) of the condensed phase of the polymer and a concentration (c_{dil}) of the dilute phase of the polymer in said system by using a following linear form equation:

$$\frac{{V}_{c o n}}{{V}_{t o t}} = \frac{1}{{c}_{c o n} - {c}_{d i l}} \cdot {c}_{tot} - \frac{{c}_{d i l}}{{c}_{c o n} - {c}_{d i l}} ,$$

E) changing at least one condition of said system and repeating the steps A) to D).

Preferably, in step E) at least one condition of said system is selected from a concentration of a component in the system wherein the component is selected from a salt, a crowding agent, or a buffer; the pH value; the pressure; or the temperature of the system, or a combination of the aforementioned conditions.

Preferably, in the method of the invention, the system is an in vitro system and selected from a solution, an emulsion, or cells.

Preferably, in the step B) of the method of present invention, the phase separation of the polymer in said system is triggered by changing: the concentration of a component in the system selected from a salt, a crowding agent, or a buffer; the pH value; the pressure; or the temperature of the system. Preferably, the salt is selected from potassium chloride, sodium chloride, magnesium chloride, or any other solute.

Preferably, in the method of the invention, when the polymer is not labelled with a fluorophore or a fluorescent protein, in step A)
the total volume (Vₜₒₜ) of said system and the total concentration (cₜₒₜ) of the polymer are determined by means of bright-field, dark-field, phase-contrast, holographic, polarization, or differential interference correlation (DIC) microscopy, or light-scattering based approaches.

Preferably, in the method of the invention, when the polymer is labelled with a fluorophore or a fluorescent protein, in the step A)
the total volume (Vₜₒₜ) of said system and the total concentration (cₜₒₜ) of the polymer are determined by means of fluorescence microscopy.

In some aspects, in step C) of the method of the present invention, step C) comprises:
C1a) encapsulating a portion of the system in an emulsion system immediately;
C2a) determining a volume (Vₑₘ) of one emulsion droplet of the emulsion system, and a condensed volume (V_{con}′) of the condensed phase of the polymer in said emulsion system;
C3a) determining a volume fraction (V_{con′}·/Vₑₘ) of the condensed volume (V_{con}′) of the condensed phase of the polymer in said emulsion system and the volume (Vₑₘ) of one emulsion droplet of said emulsion system, wherein the measured volume fraction (V_{con′}/Vₑₘ) is identical to the volume fraction (V_{con}/Vₜₒₜ) of the condensed volume (V_{con}) of the condensed phase of the polymer in the system and the total volume (Vₜₒₜ) of the system.

The emulsion system can be a water-in-oil emulsion system, an oil-in-water emulsion system or an oil-in-oil emulsion system. Preferably, the emulsion system is a water-in-oil emulsion system.

Thus, in a preferred embodiment of the method of the present invention, step C) comprises:
C1a) encapsulating a portion of the system in a water-in-oil emulsion system immediately;
C2a) determining a volume (Vₑₘ) of one emulsion droplet of the water-in-oil emulsion system, and a condensed volume (V_{con}′) of the condensed phase of the polymer in said water-in-oil emulsion system;
C3a) determining a volume fraction (V_{con′}/Vₑₘ) of the condensed volume (V_{con}′) of the condensed phase of the polymer in said water-in-oil emulsion system and the volume (Vₑₘ) of one emulsion droplet of said water-in-oil emulsion system, wherein the measured volume fraction (V_{con′}/Vₑₘ) is identical to the volume fraction (V_{con}/Vₜₒₜ) of the condensed volume (V_{con}) of the condensed phase of the polymer in the system and the total volume (Vₜₒₜ) of the system.

Preferably, when the polymer is not labelled with a fluorophore or a fluorescent protein, further comprising before step C1a):
C1a′) adding fluorescent polymer beads and/or a fluorescent protein and/or a fluorescent dye partitioning into one of the phases of the system to label the condensed phase of the polymer.

Preferably, in the step C2a), a volume (Vₑₘ) of the emulsion system, and a condensed volume (V_{con}′) of the condensed phase of the polymer in said emulsion system are determined by means of fluorescence microscopy.

Preferably, the above-described method further comprises after the step C2a):
C2b) adjusting the measured fluorescent image with a predetermined partition factor.

Preferably, in the method of the present invention, further comprising after the step B):
B′) centrifuging the system.

After triggering phase separation, many small condensates appear all over the solution and need up to hours to sediment and coarsen. Small condensates are difficult to detect. Therefore, the system is centrifuged to ensure that all condensates fuse to a big condensate in shorter time to facilitate detection.

Preferably, in the step E) of the method of the present invention, at least one condition of said system is selected from a concentration of a component in the system wherein the component is selected from a salt, a crowding agent, or a buffer; the pH value; the pressure; or the temperature of the system, or a combination of the aforementioned conditions.

In some aspects, the present invention refers to an assay method for identifying bioactive compound(s), comprising:
A) determining a total volume (Vₜₒₜ) of the system and a total concentration (cₜₒₜ) of a polymer in the system;
A′) adding a predetermined amount of test compounds into said system;
B) triggering phase separation of the polymer into a condensed phase and a dilute phase;
C) determining a volume fraction (V_{con}/Vₜₒₜ) of the condensed phase of the polymer in said system;
D) determining a concentration (c_{con}) of the condensed phase of the polymer and a concentration (c_{dil}) of the dilute phase of the polymer in said system by using a following linear form equation:

$$\frac{{V}_{c o n}}{{V}_{t o t}} = \frac{1}{{c}_{c o n} - {c}_{d i l}} \cdot {c}_{tot} - \frac{{c}_{d i l}}{{c}_{c o n} - {c}_{d i l}} ,$$

F) identifying the bioactive compounds from the test compounds; and a device comprises a plurality of systems.

Preferably, the polymer is a protein, or a polynucleic acid, preferably RNA or DNA, or a mixture of a protein and RNA, or a mixture of a protein and DNA.

Preferably, the assay method of the invention further comprises after step D):
D′) comparing the measured condensed volume (V_{con}), the measured condensed concentration (c_{con}), the measured dilute concentration (c_{dil}), and/or the measured volume fraction (V_{con}/Vₜₒₜ) of said polymer in the system with a condensed volume (V_{ref}), a condensed concentration (c_{ref}), and/or a volume fraction (V_{ref}/Vₜₒₜ) of the polymer in the presence of a reference molecule in a control system.

Thus, in some embodiments, the present invention refers to an assay method for identifying bioactive compound(s), comprising:
A) determining a total volume (Vₜₒₜ) of the system and a total concentration (cₜₒₜ) of a polymer in the system;
A′) adding a predetermined amount of test compounds into said system;
B) triggering phase separation of the polymer into a condensed phase and a dilute phase;
C) determining a volume fraction (V_{con}/Vₜₒₜ) of the condensed phase of the polymer in said system;
D) determining a concentration (c_{con}) of the condensed phase of the polymer and a concentration (c_{dil}) of the dilute phase of the polymer in said system by using a following linear form equation:

$$\frac{{V}_{c o n}}{{V}_{t o t}} = \frac{1}{{c}_{c o n} - {c}_{d i l}} \cdot {c}_{tot} - \frac{{c}_{d i l}}{{c}_{c o n} - {c}_{d i l}} ,$$

D′) comparing the measured condensed volume (V_{con}), the measured condensed concentration (c_{con}), the measured dilute concentration (c_{dil}), and/or the measured volume fraction (V_{con}/Vₜₒₜ) of said polymer in the system with a condensed volume (V_{ref}), a condensed concentration (c_{ref}), and/or a volume fraction (V_{ref}/Vₜₒₜ) of the polymer in the presence of a reference molecule in a control system;
F) identifying the bioactive compounds from the test compounds; and a device comprises a plurality of systems.

Preferably, in the assay method of the invention, the condensed volume (V_{ref}), the condensed concentration (c_{con-ref}), the dilute concentration (c_{dil-ref}) and/or the volume fraction (V_{ref}/Vₜₒₜ) of the polymer in the presence of a reference molecule is(are) predetermined or measured at the same time.

Preferably, in the assay method of the invention, the identified bioactive compound(s) increase(s)/decrease(s) the measured condensed volume (V_{con}), the measured condensed concentration (c_{con}), the measured dilute concentration (c_{dil}), and/or the measured volume fraction (V_{con}/Vₜₒₜ) of the polymer obtained by the step C) and/or D) than the predetermined condensed volume (V_{ref}), the measured condensed concentration (c_{con-ref}), the measured dilute concentration (c_{dil-ref}) and/or the measured volume fraction (V_{ref}/Vₜₒₜ) of the polymer of said polymer in the presence of a reference molecule in the control system.

Preferably, in the assay method of the invention, the device is a multi-well plate and each system is contained in each of the wells in a multi-well plate.

In some aspects, the present invention refers to use of the methods according to the invention for determining an optimal condition for crystallization of said polymer.

In some aspects, the present invention refers to a device for determining a binodal curve of a polymer in a system comprising:
i) means for measuring a total volume (Vₜₒₜ) of the system;
ii) means for measuring a volume fraction (V_{con}/Vₜₒₜ) of a condensed phase polymer in said system;
iii) means for calculating a concentration (c_{con}) of the condensed phase of the polymer and a concentration (c_{dil}) of the dilute phase of the polymer in said system by using a following linear form equation:

$$\frac{{V}_{c o n}}{{V}_{t o t}} = \frac{1}{{c}_{c o n} - {c}_{d i l}} \cdot {c}_{tot} - \frac{{c}_{d i l}}{{c}_{c o n} - {c}_{d i l}} ,$$

Preferably, the polymer is a protein, or a polynucleic acid, preferably RNA or DNA, or a mixture of a protein and RNA or a mixture of a protein and DNA.

An exemplary setup of the inventive device is shown in FIG. 10. The device utilizes the light reflection at the interface between two media of different refractive index to calculate the height of the condensed phase inside a sample container of defined volume, typically a well in a well-plate (FIG. 11).

The device comprises a light source, a dichroic mirror, a pinhole or a slit, a stage, an objective, a z-drive (a motorized focus drive), and a sample container having a defined volume. The light source is preferably an infrared light source (LED or laser) and it is directed to a high N.A objective using the dichroic mirror located behind the pinhole or the slit. At least the stage or the objective needs to be mounted on the z-drive in order to focus the light at different samples depths. In one embodiment, the stage and the objective are mounted on the z-drive. When the light is focused at the container-condensate or at the condensate-solution interfaces, light refraction will be maximal. Light intensity during the scan will be detected and the distance between the peaks corresponds to the height of the condensate phase. With the condensate phase height inside a container of known area, the volume fraction of the condensed phase can then be calculated.

### DETAILED DESCRIPTION OF THE INVENTION

### Definitions

Phase: an entity of a material system which is uniform in chemical composition and physical rate.
Phase separation: The process by which a single phase separates into two or more new phases.
Phase transition: a change in the nature of a phase or in the number of phases as a result of some variation in externally imposed conditions, such as temperature, pressure, ionic strength or activity of a component
Binodal curve: also known as coexistence curve, the locus of the compositions of two co-existing phases on a phase diagram. For example, in a temperature-composition plot the binodal curve has a maximum at the upper critical solution temperature, and/or a minimum at the lower critical solution temperature.
Phase diagram: For binary mixtures, the binodal curve is often represented by means of phase diagrams in which the system composition is plotted along the x-axis in the form of the molar fraction of one of the components as the state variable in which the coexisting phases differ. Along the y-axis, an intensive state variable, such as pressure or temperature, is plotted, which must have the same value in the coexisting phases that are in thermodynamic equilibrium. In the case of ternary mixtures, the binodal curve can be projected into a triangular diagram that represents the composition of the ternary mixture.
Tie-line: The line in a phase diagram joining the compositions of independent co-existing phases.
Condensate: the dense phase of a phase separated protein solution. “Dense phase” and “condensed phase” are synonymously used herein.
Liquid-liquid phase separation (LLPS): demixing of a protein solution into a dilute and a dense protein phase (condensates).
c_{con} (cᵢₙ): protein concentration in the dense phase after phase separation. In a phase diagram it represents the right branch of the binodal.
c_{dil} (cₒᵤₜ): protein concentration in the dilute phase after phase separation. It is also the minimum concentration required to enter the demixed state of the binodal. In a phase diagram it represents the left branch of the binodal.
Volume fraction: ratio of the volumes of the total condensed phase over the total volume of the reaction (in case of emulsions the volume of one emulsion droplet).
Water-in-oil emulsion: the resulting emulsion when an aqueous phase is mixed in the presence of an oil and a surfactant.
Labelled protein: fluorescent tag that is added to a protein. This can be directly at the expression of the protein (GFP, RFP, HALO tags plus fluorophore) or via chemical means and a fluorophore (a small fluorescent molecule, for example, Alexa fluorophores with NHS ester).
Segmentation: part of an image analysis that allows to define the regions of interest in an image (e.g. the condensates).
Polymer: The term “polymer” as used herein refers to any macromolecule consisting of repeating units, which is able to undergo liquid-liquid phase separation under specific conditions in solution, i.e. de-mixing into two distinct liquid phases (a condensed and a dilute phase) with different polymer concentrations. Suitable polymers are for example, but not limited to, proteins or polynucleic acids, including RNA or DNA.
Salts: Salts as used herein refers to any physiologically acceptable salt, including, but not limited to, sodium chloride, sodium fluoride, sodium iodide, potassium chloride, potassium iodide, potassium fluoride, magnesium chloride, calcium chloride, calcium fluoride, cesium chloride, acetate salts, and formate salts.
Crowding agents: Crowding agents as used herein are compounds that enhance phase separation of the polymer, by for instance increasing the effective polymer concentration or by changing the water activity in the system. Suitable crowding agents are for example, but not limited to, macromolecular compounds, such as PEG, dextran, Ficoll™ (an uncharged, highly branched polymer formed by the co-polymerisation of sucrose and epichlorohydrin), bovine serum albumin, or polystyrene sulfonate, or sugar based cosolvents, including trehalose, sucrose, sorbitol, and glycerol.
Buffers: Any buffer used in biochemistry is suitable for the present invention, such as tris(hydroxymethyl)aminomethane (TRIS), 4-(2-hydroxyethyl)-1-piperazineethanesulfonic acid (HEPES), 3-(N-morpholino)propanesulfonic acid (MOPS), 3-[(3-Cholamidopropyl)dimethylammonio]-1-propanesulfonate (CHAPS) or 2-(N-morpholino)ethanesulfonic acid (MES); physiological buffers whose pKs ranges from 7.37-7.42 or inorganic buffers like bicarbonate buffers or phosphate buffers.

The present invention provides a method for determining a concentration (c_{con}) of a polymer in a condensed phase and a concentration (c_{dil}) of the polymer in a dilute phase in a system comprising:
A) determining a total volume (Vₜₒₜ) of the system and a total concentration (cₜₒₜ) of the polymer;
B) triggering phase separation of the polymer into a condensed phase and a dilute phase in said system;
C) determining a volume fraction (V_{con}/Vₜₒₜ) of the condensed phase of the polymer in said system;
D) determining a concentration (c_{con}) of the condensed phase of the polymer and a concentration (c_{dil}) of the dilute phase of the polymer in said system by using a following linear form equation:

$$\frac{{V}_{c o n}}{{V}_{t o t}} = \frac{1}{{c}_{c o n} - {c}_{d i l}} \cdot {c}_{tot} - \frac{{c}_{d i l}}{{c}_{c o n} - {c}_{d i l}} ,$$

E) changing at least one condition of said system and repeating the steps A) to D).

Preferably, the polymer is selected from the group comprising or consisting of a protein, a polynucleic acid, a mixture of a protein and RNA or a mixture of a protein and DNA, a mixture of proteins, a mixture of polynucleic acids such as RNAs and DNAs, a mixture of proteins and polynucleic acids, preferably a mixture of proteins and RNAs, and a mixture of proteins and DNAs.

More preferably, the polymer is a protein, or a polynucleic acid, preferably RNA or DNA, or a mixture of a protein and RNA or a mixture of a protein and DNA.

More preferably, the polymer is a mixture of proteins, a mixture of polynucleic acids such as RNAs and DNAs, a mixture of proteins and polynucleic acids, a mixture of proteins and RNAs, and a mixture of proteins and DNAs.

In an embodiment of the present invention the method for determining a concentration (c_{con}) of a first polymer and a second polymer in a condensed phase and a concentration (c_{dil}) of the first polymer and the second polymer in a dilute phase in a system comprises:
A) determining a total volume (Vₜₒₜ) of the system and a total concentration (cₜₒₜ) of the first polymer and the second polymer;
B) triggering phase separation of the first polymer into a condensed phase and a dilute phase in said system;
C) determining a volume fraction (V_{con}/Vₜₒₜ) of the condensed phase of the first polymer and the second polymer in said system;
D) determining a concentration (c_{con}) of the condensed phase of the first polymer and the second polymer and a concentration (c_{dil}) of the dilute phase of the first polymer and the second polymer in said system by using a following linear form equation:

$$\frac{{V}_{c o n}}{{V}_{t o t}} = \frac{1}{{c}_{c o n} - {c}_{d i l}} \cdot {c}_{tot} - \frac{{c}_{d i l}}{{c}_{c o n} - {c}_{d i l}} ,$$

E) changing at least one condition of said system and repeating the steps A) to D),
   wherein the first polymer is a protein and the second polymer is a ribonucleic acid.

Reworded, in an embodiment of the present invention the method for generating a binodal curve of a first polymer and a second polymer in a system comprises:
A) determining a total volume (Vₜₒₜ) of the system and a total concentration (cₜₒₜ) of the first polymer and the second polymer;
B) triggering phase separation of the first polymer into a condensed phase and a dilute phase in said system;
C) determining a volume fraction (V_{con}/Vₜₒₜ) of the condensed phase of the first polymer and the second polymer in said system;
D) determining a concentration (c_{con}) of the condensed phase of the first polymer and the second polymer and a concentration (c_{dil}) of the dilute phase of the first polymer and the second polymer in said system by using a following linear form equation:

$$\frac{{V}_{c o n}}{{V}_{t o t}} = \frac{1}{{c}_{c o n} - {c}_{d i l}} \cdot {c}_{tot} - \frac{{c}_{d i l}}{{c}_{c o n} - {c}_{d i l}} ,$$

E) changing at least one condition of said system and repeating the steps A) to D),
   wherein the first polymer is a protein and the second polymer is a ribonucleic acid.

Preferably, in the method of the invention, the system is in vitro system and selected from a solution, an emulsion, or cells.

The term “emulsion” preferably refers to “water-in-oil emulsion” as defined above.

Preferably, in the step B) of the method of present invention, the phase separation of the polymer in said system is triggered by changing the concentration of a salt in the system, the pH value or the temperature of the system.

Preferably, in the method of the invention, when the polymer is not labelled with fluorophore or a fluorescent protein, in step A) the total volume (Vₜₒₜ) of said system and the total concentration (cₜₒₜ) of the polymer are determined by means of bright-field, dark-field, phase-contrast, holographic, polarization, or differential interference correlation (DIC) microscopy, or light-scattering based approaches.

Preferably, in the method of the invention, when the polymer is labelled with a fluorophore or a fluorescent protein, in the step A) the total volume (Vₜₒₜ) of said system and the total concentration (cₜₒₜ) of the polymer are determined by means of a fluorescence microscopy.

### Multicomponent System

In the present invention, the polymer refers to a mixture of polymers, and the mixture of polymers is selected from the group comprising or consisting of polypeptides and polynucleic acids; more preferably, the mixture of polymers is selected from a group comprising or consisting of a mixture of proteins, a mixture of RNAs, a mixture of DNAs, a mixture of proteins and RNAs, a mixture of proteins and DNAs, and a mixture of proteins, RNAs and DNAs.

Thus, in an embodiment of the present invention the method for generating a binodal curve of a mixture of polymers in a system comprises:
A) determining a total volume (Vₜₒₜ) of the system and a total concentration (cₜₒₜ) of the mixture of polymers;
B) triggering phase separation of the mixture of polymers into a condensed phase and a dilute phase in said system;
C) determining a volume fraction (V_{con}/Vₜₒₜ) of the condensed phase of the mixture of polymers in said system;
D) determining a concentration (c_{con}) of the condensed phase of the mixture of polymers and a concentration (c_{dil}) of the dilute phase of the mixture of polymers in said system by using a following linear form equation:

$$\frac{{V}_{c o n}}{{V}_{t o t}} = \frac{1}{{c}_{c o n} - {c}_{d i l}} \cdot {c}_{tot} - \frac{{c}_{d i l}}{{c}_{c o n} - {c}_{d i l}} ,$$

E) changing at least one condition of said system and repeating the steps A) to D),
   wherein the mixture of polymers is selected from the group comprising or consisting of polypeptides and polynucleic acids;
   preferably the mixture of polymers is selected from the group comprising or consisting of proteins, enzymes, antibodies, RNAs and DNAs,
   more preferably, the mixture of polymers is selected from a group comprising or consisting of a mixture of proteins, a mixture of RNAs, a mixture of DNAs, a mixture of proteins and RNAs, a mixture of proteins and DNAs, and a mixture of proteins, RNAs and DNAs.

In other words, in an embodiment of the present invention, the method for determining a concentration (c_{con}) of at least two polymers in a condensed phase and a concentration (c_{dil}) of the at least two polymers in a dilute phase in a system comprises:
A) determining a total volume (Vₜₒₜ) of the system and a total concentration (cₜₒₜ) of at least two polymers;
B) triggering phase separation of the at least two polymers into a condensed phase and a dilute phase in said system;
C) determining a volume fraction (V_{con}/Vₜₒₜ) of the condensed phase of the at least two polymers in said system;
D) determining a concentration (c_{con}) of the condensed phase of the at least two polymers and a concentration (c_{dil}) of the dilute phase of the at least two polymers in said system by using a following linear form equation:

$$\frac{{V}_{c o n}}{{V}_{t o t}} = \frac{1}{{c}_{c o n} - {c}_{d i l}} \cdot {c}_{tot} - \frac{{c}_{d i l}}{{c}_{c o n} - {c}_{d i l}} ,$$

E) changing at least one condition of said system and repeating the steps A) to D),
   wherein the at least two polymers are selected from the group comprising or consisting of polypeptides and polynucleic acids.

Preferably the at least two polymers are selected from the group comprising or consisting of proteins, enzymes, antibodies, RNAs and DNAs, more preferably, the at least two polymers are selected from a group comprising or consisting of a mixture of proteins, a mixture of RNAs, a mixture of DNAs, a mixture of proteins and RNAs, a mixture of proteins and DNAs, and a mixture of proteins, RNAs and DNAs.

Preferably, in the step B) of the method of present invention, the phase separation of the at least two polymers in said system is triggered by changing the concentration of a salt in the system, the pH value or the temperature of the system, or adding further polymers.

Preferably, in step D) of the method of the present invention, the concentration (c_{con}) of the condensed phase of the at least two polymers and the concentration (c_{dil}) of the dilute phase of each of the at least two polymers is determined by varying c_{con} or c_{dil} of said polymer while c_{con} and c_{dil} of the other polymers is kept constant, and repeating the step at different c_{con} and c_{dil} of the other polymers.

Preferably, in the method of the invention, when the at least polymers are not labelled with fluorophore or a fluorescent protein, in step A)
the total volume (Vₜₒₜ) of said system and the total concentration (cₜₒₜ) of the at least two polymers are determined by means of bright-field, dark-field, phase-contrast, holographic, polarization, or differential interference correlation (DIC) microscopy, or light-scattering based approaches.

Preferably, in the method of the invention, when the at least two polymers are labelled with a fluorophore or a fluorescent protein, in the step A)
the total volume (Vₜₒₜ) of said system and the total concentration (cₜₒₜ) of the at least two polymer are determined by means of a fluorescence microscopy.

As used herein the term “fluorophore” refers to a molecule, label or moiety that is able to absorb energy from light, to transfer this energy internally and emit said energy as light having a specific range of wavelength.

Any of the commercially available fluorophores can be used, for example, Alexa Fluor® 350, Alexa Fluor®405, Alexa Fluor®430, Alexa Fluor®488, Alexa Fluor®500, Alexa Fluor®514, Alexa Fluor®532, Alexa Fluor®546, Alexa Fluor®555, Alexa Fluor®568, Alexa Fluor®594, Alexa Fluor®610, Alexa Fluor®633, Alexa Fluor® 635, Alexa Fluor®647, Alexa Fluor®660, Alexa Fluor®680, Alexa Fluor®700, Alexa Fluor®750, fluoresceine, rhodamine B, rhodamine Red, Texas Red®, Cy3, Cy 3.5, Cy5, Cy5.5, Cy7, TRITC, FITC, ATTO-family, abberior-dyes, coumarine-derived dyes.

Preferably, the fluorophore contains any one of the following moieties (F-1)-(F-14),

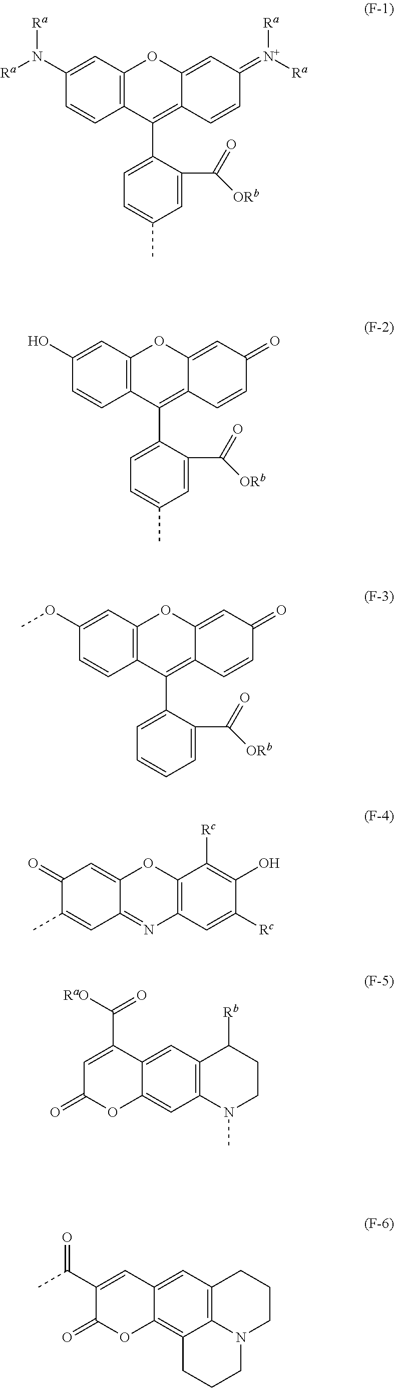

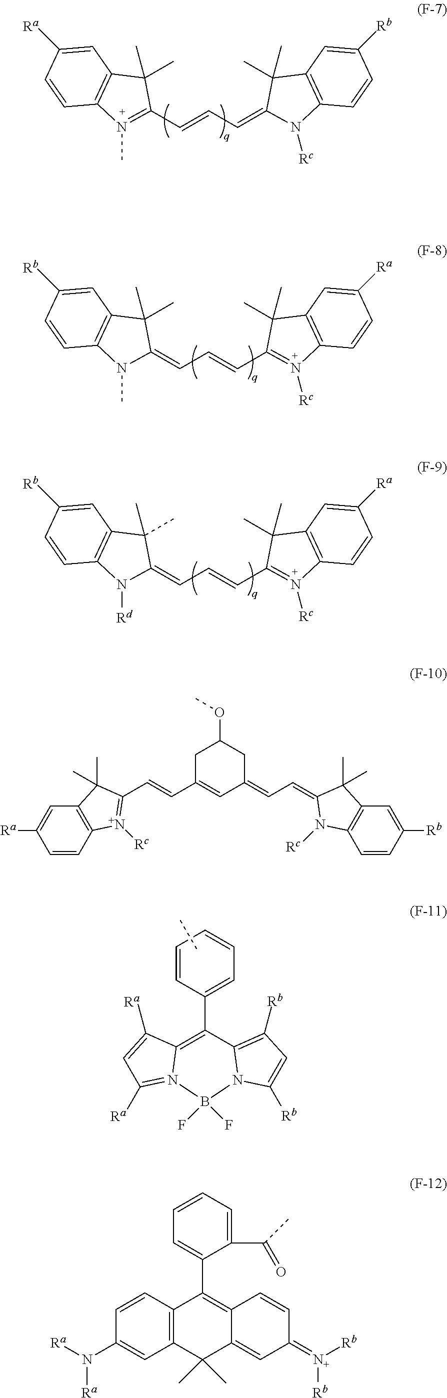

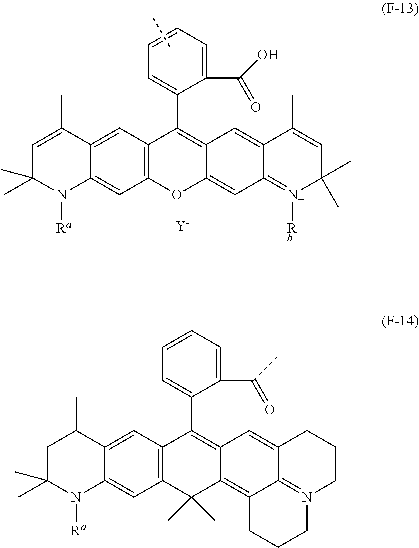

wherein R^{a} and R^{b} represent independently of each other —H, —SO₃H, —SO₃⁻, or —SO₃Na,
   R^{c} and R^{d} represent independently of each other —CH₃, —C₂H₅, —C₃H₇, —C₄Hg, —C₃H₆—SO₃⁻, or —C₄H₆—SO₃⁻;
   q is an integer selected from 1, 2 and 3.

More preferably, the fluorophore contains any one of the following moieties (F-7), (F-8), and/or (F-9):

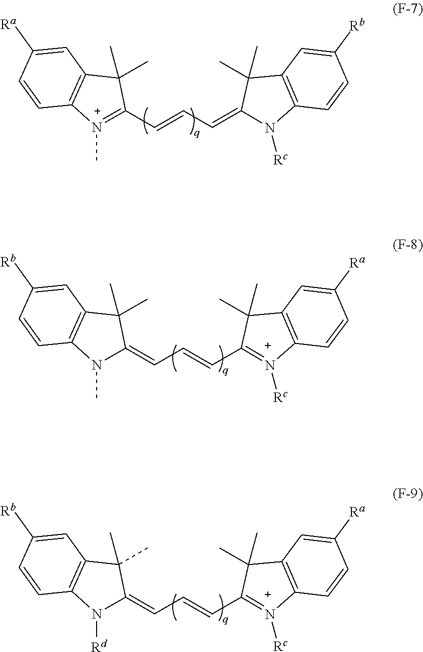

wherein R^{a} and R^{b} represent independently of each other —H, —SO₃H, —SO₃⁻, or —SO₃Na;
   R^{c} and R^{d} represent independently of each other —CH₃, —C₂H₅, —C₃H₇, —C₄H₉, —C₃H₆—SO₃—, or —C₄H₆—SO₃-q is an integer selected from 1, 2 and 3.

More preferably, the fluorophore contains any one of the moieties (F-7), (F-8), and/or (F-9), wherein R^{a} and R^{b} represent independently of each other —H; R^{c} and R^{d} represent independently of each other —CH₃, —C₂H₅, or —C₃H₇.

Still more preferably, the fluorophore contains the following moiety:

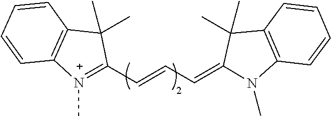

The term “a fluorescent protein” refers to a protein that absorbs light energy of a specific wavelength and re-emits light at a longer wavelength. Presented in Table 1 is a compilation of properties displayed by several of the most popular and useful fluorescent protein variants. Along with the common name and/or acronym for each fluorescent protein, the peak absorption and emission wavelengths (given in nanometers), molar extinction coefficient, and quantum yield are listed.

Preferred herein are green fluorescent proteins, such as GFP (green fluorescent protein). GFP is a versatile biological marker for monitoring physiological processes, visualizing protein localization, and detecting transgenic expression in vivo. GFP can be excited by the 488 nm laser line and is optimally detected at 510 nm

TABLE 1
Fluorescent Protein Properties

| | | | | |
|---|---|---|---|---|
| | Excitation | Emission | Molar | |
| | Maximum | Maximum | Extinction | Quantum |
| Protein | (nm) | (nm) | Coefficient | Yield |

Green Fluorescent Proteins

| | | | | |
|---|---|---|---|---|
| GFP (wt) | 395/475 | 509 | 21,000 | 0.77 |
| EGFP | 484 | 507 | 56,000 | 0.60 |
| Emerald | 487 | 509 | 57,500 | 0.68 |
| Superfolder GFP | 485 | 510 | 83,300 | 0.65 |
| Azami Green | 492 | 505 | 55,000 | 0.74 |
| mWasabi | 493 | 509 | 70,000 | 0.80 |
| TagGFP | 482 | 505 | 58,200 | 0.59 |
| TurboGFP | 482 | 502 | 70,000 | 0.53 |
| AcGFP | 480 | 505 | 50,000 | 0.55 |
| ZsGreen | 493 | 505 | 43,000 | 0.91 |
| T-Sapphire | 399 | 511 | 44,000 | 0.60 |

Blue Fluorescent Proteins

| | | | | |
|---|---|---|---|---|
| EBFP | 383 | 445 | 29,000 | 0.31 |
| EBFP2 | 383 | 448 | 32,000 | 0.56 |
| Azurite | 384 | 450 | 26,200 | 0.55 |
| mTagBFP | 399 | 456 | 52,000 | 0.63 |

Cyan Fluorescent Proteins

| | | | | |
|---|---|---|---|---|
| ECFP | 439 | 476 | 32,500 | 0.40 |
| mECFP | 433 | 475 | 32,500 | 0.40 |
| Cerulean | 433 | 475 | 43,000 | 0.62 |
| m Turquoise | 434 | 474 | 30,000 | 0.84 |
| CyPet | 435 | 477 | 35,000 | 0.51 |
| AmCyan1 | 458 | 489 | 44,000 | 0.24 |
| Midori-Ishi Cyan | 472 | 495 | 27,300 | 0.90 |
| TagCFP | 458 | 480 | 37,000 | 0.57 |
| mTFP1 (Teal) | 462 | 492 | 64,000 | 0.85 |

Yellow Fluorescent Proteins

| | | | | |
|---|---|---|---|---|
| EYFP | 514 | 527 | 83,400 | 0.61 |
| Topaz | 514 | 527 | 94,500 | 0.60 |
| Venus | 515 | 528 | 92,200 | 0.57 |
| mCitrine | 516 | 529 | 77,000 | 0.76 |
| YPet | 517 | 530 | 104,000 | 0.77 |
| TagYFP | 508 | 524 | 64,000 | 0.60 |
| PhiYFP | 525 | 537 | 124,000 | 0.39 |
| ZsYellow1 | 529 | 539 | 20,200 | 0.42 |
| mBanana | 540 | 553 | 6,000 | 0.7 |

Orange Fluorescent Proteins

| | | | | |
|---|---|---|---|---|
| Kusabira Orange | 548 | 559 | 51,600 | 0.60 |
| Kusabira Orange2 | 551 | 565 | 63,800 | 0.62 |
| mOrange | 548 | 562 | 71,000 | 0.69 |
| mOrange2 | 549 | 565 | 58,000 | 0.60 |
| dTomato | 554 | 581 | 69,000 | 0.69 |
| dTomato-Tandem | 554 | 581 | 138,000 | 0.69 |
| TagRFP | 555 | 584 | 100,000 | 0.48 |
| TagRFP-T | 555 | 584 | 81,000 | 0.41 |
| DsRed | 558 | 583 | 75,000 | 0.79 |
| DsRed2 | 563 | 582 | 43,800 | 0.55 |
| DsRed-Express (T1) | 555 | 584 | 38,000 | 0.51 |
| DsRed-Monomer | 556 | 586 | 35,000 | 0.10 |
| mTangerine | 568 | 585 | 38,000 | 0.30 |

Red Fluorescent Proteins

| | | | | |
|---|---|---|---|---|
| mRuby | 558 | 605 | 112,000 | 0.35 |
| mApple | 568 | 592 | 75,000 | 0.49 |
| mStrawberry | 574 | 596 | 90,000 | 0.29 |
| AsRed2 | 576 | 592 | 56,200 | 0.05 |
| mRFP1 | 584 | 607 | 50,000 | 0.25 |
| JRed | 584 | 610 | 44,000 | 0.20 |
| mCherry | 587 | 610 | 72,000 | 0.22 |
| HcRed1 | 588 | 618 | 20,000 | 0.015 |
| mRaspberry | 598 | 625 | 86,000 | 0.15 |
| dKeima-Tandem | 440 | 620 | 28,800 | 0.24 |
| HcRed-Tandem | 590 | 637 | 160,000 | 0.04 |
| mPlum | 590 | 649 | 41,000 | 0.10 |
| AQ143 | 595 | 655 | 90,000 | 0.04 |

In some aspects, in step C) of the method of the present invention, step C) comprises:
C1a) encapsulating a portion of the system in an emulsion system immediately;
C2a) determining a volume (Vₑₘ) of one emulsion droplet of the emulsion system, and a condensed volume (V_{con}′) of the condensed phase of the polymer in said emulsion system;
C3a) determining a volume fraction (V_{con′}/Vₑₘ) of the condensed volume (V_{con}′) of the condensed phase of the polymer in said system and the volume (Vₑₘ) of one emulsion droplet of said emulsion system, wherein the measured volume fraction (V_{con′}/Vₑₘ) is identical to the volume fraction (V_{con}/Vₜₒₜ) of the condensed volume (V_{con}) of the condensed phase of the polymer in the system and the total volume (Vₜₒₜ) of the system.

Preferably, the emulsion system is a water-in-oil emulsion system. Thus step C) of the methods described herein preferably comprises:
C1a) encapsulating a portion of the system in a water-in-oil emulsion system immediately;
C2a) determining a volume (Vₑₘ) of one emulsion droplet of the water-in-oil emulsion system, and a condensed volume (V_{con}′) of the condensed phase of the polymer in said water-in-oil emulsion system;
C3a) determining a volume fraction (V_{con′}/Vₑₘ) of the condensed volume (V_{con}′) of the condensed phase of the polymer in said water-in-oil emulsion system and the volume (Vₑₘ) of one emulsion droplet of said water-in-oil emulsion system, wherein the measured volume fraction (V_{con′}/Vₑₘ) is identical to the volume fraction (V_{con}/Vₜₒₜ) of the condensed volume (V_{con}) of the condensed phase of the polymer in the system and the total volume (Vₜₒₜ) of the system.

Preferably, in the step C2a), a volume (Vₑₘ) of the emulsion system, and a condensed volume (V_{con}′) of the condensed phase of the polymer in said emulsion system are determined by means of a fluorescence microscopy.

Preferably, the above-described method further comprises after the step C2a):
C2b) adjusting the measured fluorescent image with a predetermined partition factor.

The term “partition factor (PF)” is often used in condensate research to characterise the up-concentration of protein in the condensed phase. It can be defined by the ratio of the condensed-over dilute-phase concentration (PF=c_{con}/c_{dil}). In the condensate field it is common to determine the partition factor by comparing fluorescence intensity inside the condensate against the intensity in the dilute phase. In contrast, the inventive method allows to directly determining the partition factor by using the presented phase diagrams (FIGS. 13 and 14).

Preferably, in the method of the present invention, further comprising after the step B):
B′) centrifuging the system.

The polymer may be labeled with a fluorophore or a fluorescent protein or not.

Preferably, the height of the condensed phase film is detected/determined by means of light refraction between the surface of the condensed and solute phase. Surface detecting devices are already available in the market such as the Nikon and Olympus perfect focus and ZDC.

Alternatively, capillaries can be scanned by a laser beam to detect the surface between condensed and solute phase.

Preferably, in the step E) of the method of the present invention, the at least one condition of said system is selected from a concentration of a component in the system wherein the component is selected from a salt, a crowding agent, or a buffer; the pH value; the pressure; a crowding agent; or the temperature of the system, or a combination of the aforementioned conditions.

Salt and temperature dependent phase diagrams of two well studied proteins, fused in sarcoma (FUS) and C. elegans' P granule protein PGL-3, are demonstrated to validate the inventive method. The results are in good agreement with standard bulk methods. It is proven that phase diagrams produced by the inventive method provide novel insight into the phase behaviour of these proteins (FIG. 1). The phase diagrams are tested by temperature shift experiments that cross the derived binodal and enter the two-phase region. Finally, it was demonstrated that inventive method is accurate enough to differentiate small changes in the phase diagram caused by the addition of small molecules.

The ability to measure the (temperature dependent) binodal curve for a given polymer furthermore enables comparison to various physical theories and simulation methods. These include models like Flory-Huggins and its extensions to Voorn-Overbeek theory, field theoretic simulations, or random phase approximation. In comparing the experimental data with the predictions of these models, an appropriate physical model to describe the thermodynamics of a given phase separating polymer can be identified. Furthermore, this allows access to determining model parameters like the interaction strength between individual polymers.

Concept of the inventive method (inPhase). If a protein phase separates in a closed system, the total volume, Vₜₒₜ, is conserved and it can be expressed as the sum of the volumes of the dilute phase and the condensed phase (Vₜₒₜ=V_{dil}+V_{con}, FIG. 1c). In this closed system, the total protein mass is also conserved (mₜₒₜ=m_{dil}+m_{con}); given that the mass equals the volume times the concentration (m=V·c), we can express the previous equation as Vₜₒₜ cₜₒₜ=V_{dil} c_{dil}+V_{con} c_{con}. Rearranging the volume and mass conservation equations leads to:

$$\frac{{V}_{c o n}}{{V}_{t o t}} = \frac{1}{{c}_{c o n} - {c}_{d i l}} \cdot {c}_{tot} - \frac{{c}_{d i l}}{{c}_{c o n} - {c}_{d i l}} \qquad ( Eq . 1 )$$

Thus, it is shown that the ratio V_{con}/Vₜₒₜ, referred to as the volume fraction, changes linearly with the total protein concentration, cₜₒₜ, of the system (FIG. 1g). A linear regression to a concentration series allows to determine both c_{dil}, via the x-intercept, and c_{con} by using the slope of the line, given by (c_{con}−c_{dil})⁻¹. It therefore follows that we can determine c_{con} and c_{dil} at defined external conditions (temperature, salt, pH) by preparing samples at different total protein concentrations, cₜₒₜ, and by measuring the volume of the condensed phase, V_{con}, in a closed system of known volume, Vₜₒₜ. In principle, this allows us to measure binodal curves for ordinary binary systems. For multicomponent systems we can determine c_{con} and c_{dil} of each component by varying the concentration of said component while the concentration of the other components is kept constant. For this, we apply Eq. 1 per component, repeating this procedure at different concentrations of the other components, we can map a multidimensional phase diagram.

To set a closed environment, a water-in-oil emulsion system is used, where the aqueous solution is encapsulated in stable emulsion droplets (FIG. 1b-c). FIG. 1b shows a typical experimental scheme, where a dilution series of cₜₒₜ is mounted to a temperature-controlled stage. The volume of individual emulsion droplets can be detected from bright-field images by assuming that the droplets form nearly spherical containers (FIGS. 1d and 1e). Finally, the protein phase volume, V_{con}, is measured by image segmentation and analysis of microscopy images (FIG. 1e). The presence of tagged-protein is not strictly necessary but facilitates the segmentation.

This allows now to test the linear relationship between volume fraction and total concentration for in vitro protein solutions. The image analysis pipeline was calibrated with fluorescent polystyrene beads of known size since the inventive technique described herein relies on an accurate determination of the volume of the condensed phase (FIG. 1h). FIG. 1f shows the result of applying the method to FUS::GFP at 150 mM KCl at 15 and 25° C. The inventors observed that the linear regression is in good agreement with the experimental data.

Acquiring binodal curves with the inventive method (inPhase). The widely used fused in sarcoma (FUS) protein and PGL-3, a protein component of P granules found in the germline of C. elegans were chosen as examples to measure their temperature and salt dependent binodal curves. For PGL-3 it has been shown in vivo that the protein concentration necessary for phase separation increases with increasing temperatures (Fritsch, A. W. et al. Local thermodynamics govern formation and dissolution of Caenorhabditis elegans P granule condensates. PNAS, 2021, 118(37), e2102772118). A dilution series of PGL-3 at different temperatures recapitulates this behaviour in vitro (FIG. 2a). This representation of imaging data gives a visual impression at which protein concentration phase separation is triggered for different temperatures (indicated by a dashed line).

The binodal curves of the phase diagrams can be derived after segmentation and analysis of the microscopy data (FIG. 13 and FIG. 14). FIG. 13b-h and FIG. 14a-f show the good agreement of Eq. 1 with the experimental data and that V_{frac} increases linearly with protein concentration. The inventors found that the dilute branch concentration c_{dil} increases for higher salt concentrations as well as for higher temperatures (FIG. 13i-j and FIG. 14g-h). Comparing FUS and PGL-3 at the same salt concentration the inventors observe that FUS reaches higher concentrations in the condensed phase and its c_{dil} is more sensitive to temperature changes. The different sensitivities of both proteins to temperature could indicate a different mechanism for phase separation.

For the presented temperature phase diagrams, a critical temperature was not observed (FIG. 1a, FIG. 13i, FIG. 14g). This is because increasing the temperature results in aggregation rather than phase dissolution. This is indicated by the bending that appears on the condensed branch of the binodal towards higher protein concentrations for higher temperatures (FIG. 13i and FIG. 14g). Interestingly, the condensed branch bending is more prominent and starts at lower temperatures for higher salt concentrations. To discard artefacts from starting with cold samples and increasing the temperatures, we also measured the binodal curves starting from warm samples and cooling down.

Similar to the case for temperature, an increase in salt concentration results in an increase in c_{dil} (FIG. 13j and FIG. 14h). Here, a stronger effect of salt concentration for PGL-3 on c_{dil} compared to FUS could be seen. These findings are summarized in the 3D plot of the dilute branch surfaces against temperature and salt concentration for both proteins (FIG. 2). Furthermore, a higher sensitivity of c_{dil} to salt concentration when compared to temperature changes, especially for PGL-3 was observed. This difference can be seen by the rather box-shaped phase diagrams in FIG. 13i as compared to FIG. 13j. The different sensitivities of the phase diagrams between temperature and salt can be further used to study the molecular interactions that determine phase separation. For example, a higher sensitivity to salt as compared to temperature suggests that ionic interactions are important for phase separation in the case of PGL-3.

Testing binodals with dynamic temperature shifts. The inventors harbor the reversibility of phase separation to assess the validity of their c_{dil} estimates. For these experiments FUS was chosen because of its higher sensitivity to temperature changes. From the temperature phase diagrams of FUS at different salt concentrations, the inventors can determine a protein concentration that allows them to move in and out of the demixed region with temperature (FIG. 3a). Samples at 100, 150, 200 or 300 mM KCl that contained 1.5, 3, 5 or 10 μM of FUS, respectively were prepared. From the phase diagrams it was expected that all the samples will phase separate at similar temperatures, termed condensation temperature T_{cond}. Starting from 30° C. the temperature was reduced to 10° C. as depicted in FIG. 3b. As expected, all protein mixes started to phase separate around the predicted temperature (indicated by the black region in graph in FIG. 3b). The slightly lower T_{cond} for 300 mM KCl compared to 100 mM KCl is also reflected in a later entry into the phase separated region in the dynamic experiment. While the higher salt concentrations dissolve all condensates after heating to the initial temperature, for the lower salt concentrations small remnants are still visible after the temperature quench (small dashed outline in FIG. 3b).

It was observed that the measured dilute-branch binodals are in good agreement with the presented temperature quenches. This showcases the benefit of such a detailed understanding of a protein system for further experimental work on dynamic quenches into the demixed region.

Extension and further verification of inventive method (inPhase). Herein phase diagrams for two proteins are presented that were derived using the inventive method on a water-in-oil emulsion system. The method is however not limited to the use of emulsions that could be also generated in a microfluidic system, but can be applied wherever the volume-fraction of a condensed protein can be determined. A well-plate format was used as a scalable system that can be extended to screening approaches. In this case the total volume of the sample per well is known and the volume of the condensate phase can be determined via fluorescence. In Figure Sa, such an experiment for PGL-3 is presented, as can be seen the total condensate volume increases with increasing total protein concentration.

Next, the method in its different modes (fluorescence and brightfield segmentation in emulsions, and fluorescence segmentation in well-plates) was validated by directly measuring the protein concentration in the dilute and condensed phase via light absorbance and the BCA (bicinchoninic acid) assay (FIG. 5b, c). The results show that the inventive method outputs accurate values for both the dilute and the condensed phase concentrations. While the well-plate assay in the current iteration is less precise, the obtained average is close to the values of the other measurements. The discrepancy of the absorbance measurement for the dilute phase to all other measurements can be explained by the low protein concentrations, which lies at the detection limit of the nanoDrop device.

Partition factor and comparison to fluorescence-based quantification. The partition factor (PF) is often used in condensate research to characterise the up-concentration of protein in the condensed phase. It can be defined by the ratio of the condensed-over dilute-phase concentration (PF=c_{con}/c_{dil}). In the condensate field it is common to determine the partition factor by comparing fluorescence intensity inside the condensate against the intensity in the dilute phase. In contrast, our method allows us to directly determine the partition factor by using the presented phase diagrams (FIG. 13 and FIG. 14).

For FUS, it was observed that partition factors in the order of 10²-10³ strongly depend on the temperature and salt concentration (FIG. 15a). For PGL-3 only a small influence of temperature was observed while salt concentration can change the partition factor by two orders of magnitude between 10¹-10³ (FIG. 15a). This trend is only broken for higher temperatures and salt concentrations where a sharp increase in the partition factor takes place. It is hypothesized that these changes in partitioning factor follow from a change in protein-protein interactions and protein secondary structure.

Next, the calculated partition values are compared to the ones obtained with commonly used quantitative fluorescence intensity approach (FIG. 15b). Given that fluorescence was also used to segment the condensate volume, the same data can be used to directly compare both methods (PF_{intensity} via intensity, PF_{inPhase} via inventive method). The inventors found a strong dependence of the quantitative intensity-derived partition factor PF_{intensity} on condensate size for both FUS and PGL-3 (FIG. 15b). Above a condensate volume of approximately 10² μm³ the partition factor PF_{intensity} levels off to a plateau-like value that is however still smaller than the value the inventors calculated via the method disclosed herein (FIG. 15b, dashed line). A value for PF_{intensity} for a given external condition can be calculated by only using condensates above a threshold volume that is in the plateau region. In general, the PF_{intensity} underestimates protein partitioning (FIG. 15c). This effect is likely caused by an erroneous estimation of the fluorescence intensity inside the condensate phase. These could be caused by limitations of the optical system as well as by a different chemical environment inside the phase that changes the fluorescence properties. To this end, the inventors tested the influence of the fraction of labelled protein on the partition factor PF_{intensity} and PF_{inPhase} for PGL-3. While an increase in the partition factor is seen for increased fraction of labelled protein (FIG. 15d), the ratio PF_{I}/PF_{C} is constant over all label fractions tested (FIG. 15d).

### Discussion

Phase diagrams produced by the inventive method that present the dilute and condensed phase protein concentrations have been reported for phase separating proteins. These phase diagrams contain rich information on the protein behaviour and interactions that can be used to assess current theoretical approaches (cite later). Yet, to realize the full potential of phase separation research better methods for measuring phase diagrams are still required. The inventive method offers a powerful approach to address this challenge. The inventors showed quantitatively that accurate phase diagrams can be generated at defined temperature and salt concentrations for FUS and PGL-3 proteins. Based on these phase diagrams dynamic temperature experiments can be performed to assess the reversibility of phase separation and quantify the effect of small molecules on phase behaviour.

The inventive method is based on mass- and volume-conversation to measure accurate concentrations of the dilute and condensed phase of phase separating proteins. The underlying principles and techniques make this method accessible to the research community because it relies on commonly available equipment (fluorescent or bright field microscope), modest computational power and low amounts of protein. The inventive approach does not necessitate the use of labelled protein, which not only reduces the time required for cloning and protein purification but also removes artefacts associated with the use of fluorescent tags. Furthermore, the accurate measurement of binodal curves with the inventive method has the potential to increase reproducibility in the protein phase separation community as protein concentrations in and out of the phase can be reported with their experimental errors.

It was demonstrated that the partition factors calculated using the inventive method overcome common issues encountered when using quantitative fluorescence microscopy. For example, given a 16-bit image of a droplet with a fluorescence count of 65×10³ in an image that has a dark current signal of 100 counts and with a fluorescent intensity outside the condensate of 150 counts, the maximum partition factor that can be detected is about 1.3×10³. As it has been shown, this range is not enough to measure the partitioning factor of FUS, and probably extends to other proteins of the same family. The dynamic range of the camera is not the only limiting factor in quantitative fluorescence but it is well known that fluorescent properties depend on the environment. Given that the chemical environment is different inside the condensate compared to the dilute phase it is not safe to assume a linear relationship of the fluorescent intensity between the condensate and its surroundings.

Other known methods to study the dilute or condensed branch concentrations include more elaborate bulk measurements, measurements of optical density, microfluidic systems, or fluorescence correlation spectroscopy (FCS). While optical density measurements provide a reliable way to determine the condensed branch concentrations as well as a limited possibility to be used in an in vivo setting, they do not provide dilute branch concentrations, need dedicated equipment, and are difficult to be converted to be used for screening.

In conclusion, the inventive method is an efficient new method for measuring binodal curves, and it will contribute to developing the study of phase separating proteins. While other techniques have been developed to measure binodal curves, they often require specialised equipment (microfluidic chips) or great amounts of protein and therefore their applicability might be limited. The concept at the core of inventive method is readily accessible to a wide community and that the experimental workflow, performing a protein titration, can be readily adapted.

### Effect of Small Molecule Drugs and Screening Methods

The methods of the present invention are compared to the widely used quantitative fluorescence approaches and find that these methods underestimate the protein concentration in the condensates for FUS and PGL-3. Thus, the inventive methods can be used in large-scale screening approaches to generate databases of binodal curves, opening the possibility for the thermodynamic assessment of entire protein families and for pharmaceutical studies.

In some aspects, the present invention refers to an assay method for identifying bioactive compound(s), comprising:
A) determining a total volume (Vₜₒₜ) of the system and a total concentration (cₜₒₜ) of a polymer in the system;
A′) adding a predetermined amount of test compounds into said system;
B) triggering phase separation of the polymer into a condensed phase and a dilute phase
C) determining a volume fraction (V_{con}/Vₜₒₜ) of the condensed phase of the polymer in said system;
D) determining a concentration (c_{con}) of the condensed phase of the polymer and a concentration (c_{dil}) of the dilute phase of the polymer in said system by using a following linear form equation:

$$\frac{{V}_{c o n}}{{V}_{t o t}} = \frac{1}{{c}_{c o n} - {c}_{d i l}} \cdot {c}_{tot} - \frac{{c}_{d i l}}{{c}_{c o n} - {c}_{d i l}} ,$$

F) identifying the bioactive compounds from the test compounds;
wherein the polymer is a protein, or a polynucleic acid; and a device comprises a plurality of systems.

Preferably, the assay method of the invention further comprises after step D):
D′) comparing the measured condensed volume (V_{con}), the measured condensed concentration (c_{con}), and/or the measured volume fraction (V_{con}/Vₜₒₜ) of said polymer in the system with a condensed volume (V_{ref}), a condensed concentration (c_{ref}), and/or a volume fraction (V_{ref}/Vₜₒₜ) of the polymer in the presence of a reference molecule in a control system.

In some embodiments, the present invention thus refers to an assay method for identifying bioactive compound(s), comprising:
A) determining a total volume (Vₜₒₜ) of the system and a total concentration (cₜₒₜ) of a polymer in the system;
A′) adding a predetermined amount of test compounds into said system;
B) triggering phase separation of the polymer into a condensed phase and a dilute phase
C) determining a volume fraction (V_{con}/Vₜₒₜ) of the condensed phase of the polymer in said system;
D) determining a concentration (c_{con}) of the condensed phase of the polymer and a concentration (c_{dil}) of the dilute phase of the polymer in said system by using a following linear form equation:

$$\frac{{V}_{c o n}}{{V}_{t o t}} = \frac{1}{{c}_{c o n} - {c}_{d i l}} \cdot {c}_{tot} - \frac{{c}_{d i l}}{{c}_{c o n} - {c}_{d i l}} ,$$

D′) comparing the measured condensed volume (V_{con}), the measured condensed concentration (c_{con}), and/or the measured volume fraction (V_{con}/Vₜₒₜ) of said polymer in the system with a condensed volume (V_{ref}), a condensed concentration (c_{ref}), and/or a volume fraction (V_{ref}/Vₜₒₜ) of the polymer in the presence of a reference molecule in a control system;
F) identifying the bioactive compounds from the test compounds;
   wherein the polymer is a protein, or a polynucleic acid; and a device comprises a plurality of systems.

Preferably, in the assay method of the invention, the condensed volume (V_{ref}), the condensed concentration (c_{ref}), and/or the volume fraction (V_{ref}/Vₜₒₜ) of the polymer in the presence of a reference molecule is(are) predetermined or measured at the same time.

Preferably, in the assay method of the invention, the identified bioactive compound(s) increase(s) the measured condensed volume (V_{con}), the measured condensed concentration (c_{con}), the measured dilute concentration (c_{dil}), and/or the measured volume fraction (V_{con}/Vₜₒₜ) of the polymer obtained by the step C) and/or D) than the predetermined condensed volume (V_{ref}), the measured condensed concentration (c_{ref}), and/or the measured volume fraction (V_{ref}/Vₜₒₜ) of the polymer of said polymer in the presence of a reference molecule in the control system.

Preferably, in the assay method of the invention, the device is a multi-well plate and each system is contained in each of wells in a multi-well plate.

The bioactive compound includes, but not limited to small molecules, peptides, petidomimetics, proteins, antibodies such as monoclonal antibodies, and nucleic acids such as DNA, and RNA.

The bioactive compound is preferably a small molecule (molecular weight<1000 Da), and more preferably a therapeutically active small molecule, i.e. a small molecule drug.

To demonstrate the possibilities of drug screening, a small molecule (for example, Lipoamide) is used that has been indicated to affect the phase separation of FUS a protein related to ALS. It has so far been difficult to accurately the extent of the effect because of a lack of quantitative measures.

Example 3 shows the effect of lipoamide on the phase behavior of FUS where a reduced amount of conformational changes at higher temperatures was observed. This is indicated by the earlier bent of the condensed phase branch of the binodal for the untreated control (DMSO). This can also be seen in the partition factor c_{dil}/c_{con}.

### Use of Phase Diagrams for Determining Optimal Phase Separation Conditions

In some aspects, the present invention refers to use of the methods according to the invention for determining an optimal condition for crystallization of said polymer.

The binodal curve is the coexistence curve for thermodynamic stability which a transition occurs from a stable single-phase mixture towards loss of miscibility and subsequent demixing. This results in the formation of a dilute and condensed (dense) polymer liquid phase. Liquid-liquid demixing involves a nucleation step, and as such, requires the passage over an activation barrier.

The spinodal curve describes the limit or loss of this metastability, i.e. barrier free phase separation and instability against all fluctuations. The spinodal curve is of kinetic origin. It defines the transition to a nucleus size of one polymer and has been observed, in particular for proteins. The liquid-liquid critical point is then the intersection of the binodal and spinodal curves. At the critical point, there is no distinction between the two liquid phases and phase boundaries cease to exist. Liquid-liquid demixing has been observed experimentally for many proteins. For these cases, the liquid-liquid-coexistence curve is located in the metastable region under the liquid-solid coexistence curve.

Thus, the phase diagram derived from the binodal curves produced by the methods according to the invention is useful for determining an optimal condition for phase separation of said polymer, in particular, proteins.

Preferably, the polymer is a protein, or a polynucleic acid, preferably RNA or DNA, or a mixture of a protein and RNA or a mixture of a protein and DNA, a mixture of proteins, a mixture of polynucleic acids such as RNAs or DNAs, or a mixture of proteins and polynucleic acids, preferably a mixture of proteins and RNAs, or a mixture of proteins and DNAs.

### Phase Diagrams In Vivo

In general, the method of the present invention can be used for any phase separating system. This is also true for in vivo settings of phase separating proteins. The natural or induced fluctuations in the total protein concentration of cells are equivalent to protein concentration titration series of the inventors. Thus, the inventive method can be applied to calculate c_{con} and c_{dil}. As previously discussed, we determine the volume fraction of the phase and the total concentration of protein (e.g. via GFP labelled protein). This is shown exemplary for C. elegans embryos and the proteins PGL-1 and PGL-3 that are constitutive for P granules (FIG. 8).

This exemplifies the ability of the inventive method to study multicomponent systems, e.g. by titrating the concentration of one component while keeping the other constant. Furthermore in vivo screens could be used in translational research for drug discovery.

The present invention is directed to an in vivo method for the diagnosis of a disease associated with and/or caused by phase separation of a polymer in a cell-containing sample comprising:
A) determining a total concentration (cₜₒₜ) of a phase-separated polymer within a cell contained in said sample;
C) determining a volume fraction (V_{con}/Vₜₒₜ) of a condensed phase of the polymer in said cell contained in said sample;
D) determining a concentration (c_{con}) of the condensed phase of the polymer and a concentration (c_{dil}) of the dilute phase of the polymer in said cell contained in said sample by using a following linear form equation:

$$\frac{{V}_{c o n}}{{V}_{t o t}} = \frac{1}{{c}_{c o n} - {c}_{d i l}} \cdot {c}_{tot} - \frac{{c}_{d i l}}{{c}_{c o n} - {c}_{d i l}} ,$$

E) repeating steps A)-D) on a different cell contained in said sample.

Preferably, the polymer is a protein, or a polynucleic acid, preferably RNA or DNA, or a mixture of a protein and RNA or a mixture of a protein and DNA, a mixture of proteins, a mixture of polynucleic acids such as RNAs or DNAs, or a mixture of proteins and polynucleic acids, preferably a mixture of proteins and RNAs, or a mixture of proteins and DNAs.

More preferably, the polymer is a protein, or a polynucleic acid, preferably RNA or DNA, or a mixture of a protein and RNA or a mixture of a protein and DNA,

Preferably, the disease associated with and/or caused by phase separation of a polymer is cancer, a neurodegenerative disease or an infectious disease. More preferably, the disease is selected from ALS, Parkinson, Alzheimer, Huntington, frontotemporal dementia, cancer, and infectious disease.

A phase-separated polymer refers to a polymer being present in condensed phase and a dilute phase within the system.

In a preferred embodiment the in vivo method for the diagnosis of a disease associated with and/or caused by phase separation of a polymer in a cell-containing sample comprising:
A) determining a total concentration (cₜₒₜ) of a phase-separated polymer within a cell contained in said sample;
C) determining a volume fraction (V_{con}/Vₜₒₜ) of a condensed phase of the polymer in said cell contained in said sample;
D) determining a concentration (c_{con}) of the condensed phase of the polymer and a concentration (c_{dil}) of the dilute phase of the polymer in said cell contained in said sample by using a following linear form equation:

$$\frac{{V}_{c o n}}{{V}_{t o t}} = \frac{1}{{c}_{c o n} - {c}_{d i l}} \cdot {c}_{tot} - \frac{{c}_{d i l}}{{c}_{c o n} - {c}_{d i l}} ,$$

E) inducing RNA interference to decrease the total concentration (cₜₒₜ) of a phase-separated polymer and repeating steps A)-D),
   wherein the polymer is a protein.

In a preferred embodiment the in vivo method for the diagnosis of a disease associated with and/or caused by phase separation of a polymer in a cell-containing sample comprising:
A) determining a total concentration (cₜₒₜ) of a phase-separated polymer within a cell contained in said sample;
C) determining a volume fraction (V_{con}/Vₜₒₜ) of a condensed phase of the polymer in said cell contained in said sample;
D) determining a concentration (c_{con}) of the condensed phase of the polymer and a concentration (c_{dil}) of the dilute phase of the polymer in said cell contained in said sample by using a following linear form equation:

$$\frac{{V}_{c o n}}{{V}_{t o t}} = \frac{1}{{c}_{con} - {c}_{dil}} \cdot {c}_{tot} - \frac{{c}_{dil}}{{c}_{con} - {c}_{dil}} ,$$

E) inducing gene expression in said cell to increase the total concentration (cₜₒₜ) of a phase-separated polymer and repeating steps A)-D),
   wherein the polymer is a protein.

The present invention is directed to an ex vivo method for the diagnosis of a disease associated with and/or caused by phase separation of a polymer in a cell-containing sample comprising:
A1) obtaining a cell-containing sample from a subject,
A2) determining a total concentration (cₜₒₜ) of a phase-separated polymer within a cell contained in said sample;
C) determining a volume fraction (V_{con}/Vₜₒₜ) of a condensed phase of the polymer in said cell contained in said sample;
D) determining a concentration (c_{con}) of the condensed phase of the polymer and a concentration (c_{dil}) of the dilute phase of the polymer in said cell contained in said sample by using a following linear form equation:

$$\frac{{V}_{c o n}}{{V}_{t o t}} = \frac{1}{{c}_{con} - {c}_{dil}} \cdot {c}_{tot} - \frac{{c}_{dil}}{{c}_{con} - {c}_{dil}} ,$$

E) repeating steps A)-D) on a different cell contained in said sample.

Preferably, the polymer is a protein, or a polynucleic acid, preferably RNA or DNA, or a mixture of a protein and RNA or a mixture of a protein and DNA.

Preferably, the disease associated with and/or caused by phase separation of a polymer is cancer, a neurodegenerative disease or an infectious disease. More preferably, the disease is selected from ALS, Parkinson, Alzheimer, Huntington, frontotemporal dementia, cancer, and infectious disease.

In a preferred embodiment the ex vivo method for the diagnosis of a disease associated with and/or caused by phase separation of a polymer in a cell-containing sample comprising:
A1) obtaining a cell-containing sample from a subject,
A2) determining a total concentration (cₜₒₜ) of a phase-separated polymer within a cell contained in said sample;
C) determining a volume fraction (V_{con}/Vₜₒₜ) of a condensed phase of the polymer in said cell contained in said sample;
D) determining a concentration (c_{con}) of the condensed phase of the polymer and a concentration (c_{dil}) of the dilute phase of the polymer in said cell contained in said sample by using a following linear form equation:

$$\frac{{V}_{c o n}}{{V}_{t o t}} = \frac{1}{{c}_{con} - {c}_{dil}} \cdot {c}_{tot} - \frac{{c}_{dil}}{{c}_{con} - {c}_{dil}} ,$$

E) inducing RNA interference to decrease the total concentration (cₜₒₜ) of a phase-separated polymer and repeating steps A)-D),
   wherein the polymer is a protein.

In a preferred embodiment the ex vivo method for the diagnosis of a disease associated with and/or caused by phase separation of a polymer in a cell-containing sample comprising:
A1) obtaining a cell-containing sample from a subject,
A2) determining a total concentration (cₜₒₜ) of a phase-separated polymer within a cell contained in said sample;
C) determining a volume fraction (V_{con}/Vₜₒₜ) of a condensed phase of the polymer in said cell contained in said sample;
D) determining a concentration (c_{con}) of the condensed phase of the polymer and a concentration (c_{dil}) of the dilute phase of the polymer in said cell contained in said sample by using a following linear form equation:

$$\frac{{V}_{c o n}}{{V}_{t o t}} = \frac{1}{{c}_{con} - {c}_{dil}} \cdot {c}_{tot} - \frac{{c}_{dil}}{{c}_{con} - {c}_{dil}} ,$$

E) inducing gene expression in said cell to increase the total concentration (cₜₒₜ) of a phase-separated polymer and repeating steps A)-D),
   wherein the polymer is a protein.

Suitable samples for the diagnostic methods described herein are for example, but not limited to, bodily fluids, including blood, urine, cerebrospinal fluid, peritoneal fluid, pleural fluid, ascitic fluid, blood plasma, liver extracts and interstitial fluid.

### Phase Volume Detection Device

The presented approaches to determine the volume fraction of the condensed phase rely on the use of imaging and image segmentation. This has the intrinsic problem of time and cost inefficiency. Herein, alternative approaches to determine the volume fraction using light scattering properties of a surface are presented.

Thus, the present invention is directed to a device for determining a binodal curve of a polymer in a system comprising:
i) means for measuring a total volume (Vₜₒₜ) of the system;
ii) means for measuring a volume fraction (V_{con}/Vₜₒₜ) of a condensed phase of the polymer in said system;
iii) means for calculating a concentration (c_{con}) of the condensed phase of the polymer and a concentration (c_{dil}) of the dilute phase of the polymer in said system by using a following linear form equation:

$$\frac{{V}_{c o n}}{{V}_{t o t}} = \frac{1}{{c}_{con} - {c}_{dil}} \cdot {c}_{tot} - \frac{{c}_{dil}}{{c}_{con} - {c}_{dil}} ,$$

wherein the polymer is a protein, or a polynucleic acid.

Using well-plates that are high binding towards the protein we can generate a film of condensed phase after centrifugation (see FIG. 6). Thus, a volume fraction (V_{con}/Vₜₒₜ) of a condensed phase of the polymer in said system can be determined by detecting the height of the condensed phase film by means of light refraction between the surface of the condensed and solute phase. Thus, surface detecting devices such as the Nikon and Olympus perfect focus and ZDC can be used as means for measuring a volume fraction (V_{con}/Vₜₒₜ) of a condensed phase of the polymer in said system.

An exemplary setup of the inventive device is shown in FIG. 10. The device utilizes the light reflection at the interface between two media of different refractive index to calculate the height of the condensed phase inside a sample container of defined volume, typically a well in a well-plate (see FIG. 11).

The device comprises a light source, a dichroic mirror, a pinhole or a slit, a stage, an objective, a z-drive (a motorized focus drive), and a sample container having a defined volume. The light source is preferably an infrared light source (LED or laser) and it is directed to a high N.A objective using the dichroic mirror located behind the pinhole or the slit. At least the stage or the objective needs to be mounted on the z-drive in order to focus the light at different samples depths. In one embodiment, the stage and the objective are mounted on the z-drive. When the light is focused at the container-condensate or at the condensate-solution interfaces, light refraction will be maximal. Light intensity during the scan will be detected and the distance between the peaks corresponds to the height of the condensate phase (FIG. 11). With the condensate phase height inside a container of known area, the volume fraction of the condensed phase can then be calculated.

Preferably, the sample container has a constant cross-section in height for facilitating the determination of the liquid level and height of the condensed phase film. Preferably, the sample container is of cylindrical shape.

Alternatively, capillaries or capillary tubings or capillary tubes can be used as sample containers. The capillary tubes are filled with the phase separated solution and scanned by a laser beam to detect the surface between condensed phase and solute phase. Preferably, the capillary tubes are centrifuged prior scanning to coalesce the condensed phase into one phase.

Thus, in one embodiment, the device comprises a laser light source, a dichroic mirror, a pinhole or a slit, a stage, an objective, a z-drive (a motorized focus drive), and capillary tubes. The light source is directed to a high N.A objective using the dichroic mirror located behind the pinhole or the slit.

The means for calculating a concentration (c_{con}) of the condensed phase of the polymer and a concentration (c_{dil}) of the dilute phase of the polymer in said system is preferably a computer which is connected to a controller and a detector, wherein the computer is configured to receive signals detected from the detector and wherein the computer is configured to calculate a concentration (c_{con}) of the condensed phase of the polymer and a concentration (c_{dil}) of the dilute phase of the polymer in said system by using a following linear form equation:

$$\frac{{V}_{c o n}}{{V}_{t o t}} = \frac{1}{{c}_{con} - {c}_{dil}} \cdot {c}_{tot} - \frac{{c}_{dil}}{{c}_{con} - {c}_{dil}} .$$

### DESCRIPTION OF FIGURES

FIG. 1: Concentration series in defined volumes allow for determination of c_{con} and c_{dil}. a, Schematic of the binodal of a phase diagram with dilute branch cₒᵤₜ and condensed branch c_{con} concentrations connected via a tie line. b, Schematic of the experimental protocol and setup design using a titration of cₜₒₜ in water-in-oil emulsions mounted on a temperature-controlled device. c, The total concentration of protein cₜₒₜ used in the volume Vₜₒₜ of an emulsion droplet separates into the dilute phase with c_{dil} and Vₒᵤₜ and the condensed phase with c_{con} and Vᵢₙ. d, Example images of PGL-3 condensates in emulsions mounted on the device in b. The brightfield channel is used to determine the volume of the emulsions droplets while the fluorescent channel is used to determine the condensed phase volume (scale bar 100 μm). e, Zoom on segmentation of emulsion droplet and condensate of white square region in d (scale bar 10 μm). f, Using mass and volume conservation, the volume fraction of the condensed phase (V_{con}/Vₜₒₜ) describes a linear form with the slope (c_{con}−c_{dil})⁻¹ and an offset of c_{dil}/(c_{con}-_{dil}), allowing to derive both c_{dil} and c_{con} via a linear regression against cₜₒₜ. g, equation 1 and linear regression of the ratio Vᵢₙ/Vₜₒₜ and the total polymer concentration, cₜₒₜ. h, calibration of image analysis pipeline with fluorescent polystyrene beads of known size.

FIG. 2: Salt and temperature dependence of phase separated FUS and PGL-3 protein. a, False colour experimental images of PGL-3+5% PGL-3::GFP for various temperatures and concentrations (binodal line is a guide to the eye). B, Temperature vs. protein concentration phase diagrams of dilute (c_{dil}) and condensed (c_{con}) branch of FUS::GFP for various salt concentration. Derived from linear regression to cₜₒₜ concentration series for each salt concentration and temperature (error bars are SD). c, Temperature vs. protein concentration phase diagrams for PGL-3 spiked with 5% PGL-3::GFP. d, e, Data of b and c presented as salt concentration vs. protein concentration phase diagrams for FUS and PGL-3 respectively. f, Comparison of the dilute branch concentrations c_{dil} of FUS and PGL-3 as surfaces in a salt concentration, temperature space.

FIG. 3: Reversible temperature quenches in and out of the two-phase region for FUS::GFP. a, Dilute branch binodal (c_{dil}) for FUS::GFP at different salt concentrations. Solid dots demark the condensation temperature T_{cond} for the protein concentrations used at specific salt concentrations that allow to cross the binodal with a temperature quench. b, Timeseries of temperature quenches from 35° C. to 10° C. and back to 35° C. Upper panel shows experimental data for the volume fraction and measured temperature curves for four different salt and protein concentrations of FUS::GFP (10 s resolution). Lower panel shows example projections of 3D image stacks at specific time points (also indicated by the red dots on the temperature curve). Yellow circles indicate remaining condensates/aggregates for low salt concentrations after the temperature quench.

FIG. 4: Effect of temperature and salt on the partition factor and comparison to quantitative fluorescence. a, b, Partition factor PF_{c} (c_{con}/c_{dil}) derived via inventive method (inPhase) for various temperatures and salt concentrations for FUS::GFP and PGL-3+5% PGL-3::GFP respectively (y-axis logarithmic). c, Partition factor as a function of the amount of labelled PGL-3 protein fraction. While PF_{c} is derived via inPhase PF_{I} is the partition factor calculated via fluorescence intensities in the dilute and condensed phase of the same sample. d, Partition factor PF_{I} of individual condensates as a function of their volume for FUS::GFP and GFP and PGL-3+5% PGL-3::GFP respectively. Upper and lower panels contain the same data with the lower panels depicted with a logarithmic x-axis (dashed lines indicate PF_{C} as derived via inPhase method for the same data-set). e, Relative partition factor of inPhase PF_{c} divided by quantitative intensity-based PF_{I} for various salt concentrations and temperatures for FUS and PGL-3.

FIG. 5: Comparison of variations of the inventive method (inPhase) with standard bulk measurements for PGL-3. a, Overview of cₜₒₜ titration series of projections of 3D image stacks for PGL-3+5% PGL-3::GFP in 386 well-plates. b, Comparison of dilute branch concentrations c_{dil} for inPhase using fluorescence (fluo) and brightfield (BF) in emulsions and the well-plate approach versus bulk measurements via Pierson BCA (BCA) and 280 nm UV absorption measurements in a nanoDrop (nDrop). c, Comparison of condensed branch concentrations c_{con} for inPhase and bulk approaches. Dashed line indicates the value for the emulsion fluorescence-based evaluation as a standard.

FIG. 6: shows an exemplary setup for the determination of the height of a condensed phase layer within a container (no need for fluorescent labels). This representation uses the reflection of light at the interface to determine the thickness of a layer.

FIG. 7: shows the time-dependent changes of condensed and solute concentration for two exemplary proteins.

c_{dil} and c_{con} do not change considerably during the experimental window. a, b, Time dependent evaluation using the inventive method of dilute and condensed branch concentrations for PGL-3 (a) and FUS (b) (Errors from error propagation of linear regression confidence intervals). Shaded areas depict the time-window used for the temperature and salt dependent phase diagrams.

FIG. 8: Protein concentration heterogeneity in C. elegans embryos allows to test the inventive method in vivo. a, Data of condensed volume fraction against total PGL-1::GFP concentration at 20° C. (dots, N=46). Linear regression to the data (dashed line) allows to use equation 1 and derive c_{dil} and effective c_{cond} concentrations. b, Data of condensed volume fraction against total PGL-3::GFP concentration at 20° C. (dots, N=29). Linear regression to the data (dashed line) allows to use equation 1 and to derive c_{dil} and effective c_{cond} concentrations. c, scheme of a one-cell C. elegans embryo. d, Temperature dependent phase diagram of PGL-1::GFP in C. elegans embryos. Adapted from Fritsch et al. PNAS, 2021, 118(37), e2102772118.

FIG. 9: shows comparison of the effect of untreated control (DMSO) and Lipoamide on the phase diagram (top panel) and partition factor of FUS (bottom panel).

FIG. 10: shows a schematic drawing of the device according to the invention to determine V_{frac} using the difference in refractive index.

FIG. 11: shows a diagram of the intensity of reflected light in dependency of the sample depth.

FIG. 12: RNA to protein ratio dependence of phase separated FUS. a, molar RNA to protein ratio vs. total concentration (RNA+protein) phase diagrams of dilute (c_{dil}) and condensed (c_{con}) branch of FUS::GFP for various RNA to protein ratios. Derived from linear regression to cₜₒₜ concentration series for each RNA to protein ratio; b, volume fraction vs. total concentration (RNA+protein) at a constant molar RNA to protein ratio of 0.01. Experiments were carried out as described in Example 5 with Poly(A) RNA as RNA and FUS-GFP as protein.

FIG. 13: Linear regressions of V_{frac}(cₜₒₜ) data and phase diagrams for FUS. a, False colour experimental images of PGL-3+5% PGL-3::GFP for various temperatures and concentrations (binodal line is a guide to the eye). b-g, Linear regressions of V_{frac}(cₜₒₜ) of samples prepared at 100 (b), 150 (c), 175 (d), 200 (e), 225 (f) and 240 (g) mM KCl and measured at different temperatures. Experimental data is pooled from all independent repeats and then fitted to Equation 1. Each individual experiment consists of data points from many emulsion droplets. Error bars depict standard deviations. h, Quality of linear regression (R-squared value) of the data presented in panel b-g. Shaded areas depict standard deviations derived from individual repeats. h, i, Temperature (i) and salt (j) phase diagrams presenting the derived data for c_{con} and c_{dil} from the linear regressions in panel b-g. Lines connecting the data points are a guide to the eye. Shaded areas depict the errors derived from propagation of confidence intervals of the linear regression in panel b-g.

FIG. 14: Linear regressions of V_{frac}(cₜₒₜ) data and phase diagrams for FUS. a-e, Linear regressions of V_{frac}(cₜₒₜ) of samples prepared at 75 (a), 100 (b), 150 (c), 200 (d) and 300 (e) mM KCl and measured at different temperatures. Experimental data is pooled from all independent repeats and then fitted to Eq. 1. Each individual experiment consists of datapoints from many emulsion droplets. Error bars depict standard deviations. f, Quality of linear regression (R-squared value) of data presented in panel a-e. Shaded areas depict standard deviations derived from individual repeats. g, h, Temperature (g) and salt (h) phase diagrams presenting the derived data for c_{con} and c_{dil} from the linear regressions in panel a-e. Lines connecting the data points are a guide to the eye. Shaded areas depict the errors derived from propagation of confidence intervals of the linear regression in panel a-e.

FIG. 15: Effect of temperature and salt on the partition factor and comparison to quantitative fluorescence for FUS and PGL-3. a, Partition factor PF_{inPhase} (c_{con}/c_{dil}) derived via inPhase method for various temperatures and salt concentrations for FUS::GFP (left panel) and PGL-3+5% PGL-3::GFP (right panel). b, PF_{intensity} of individual condensates as a function of their volume for FUS::GFP (left panel) and PGL-3+5% PGL-3::GFP (right panel). Upper and lower panels contain the same data with the lower panels using a logarithmic x-axis (dashed lines indicate PF_{inPhase} for the same data-set). c, Ratio PF_{inPhase}/PF_{intensity} for various salt concentrations and temperatures for FUS (left panel) and PGL-3 (right panel). d, Partition factor (PF) as a function of the amount of labelled PGL-3 protein fraction. The upper panel shows the c_{con} (dark gray) and c_{dil} (light gray) calculated using inPhase. The lower panel shows the ratio of PF_{inPhase}/PF_{intensity} at different GFP percentages. PF_{intensity} underestimates the partition factor. Error bars show SD.

FIG. 16: Increasing the RNA to protein ratio reduces c_{dil}. a, Titration of total protein concentration at a constant ratio of poly(A) RNA to protein concentration. R-squared values are from linear regression to the individual curves (errors depict standard deviation). b-c, Derived values of c_{dil} and effective c_{con} using inPhase on the data presented in panel a.

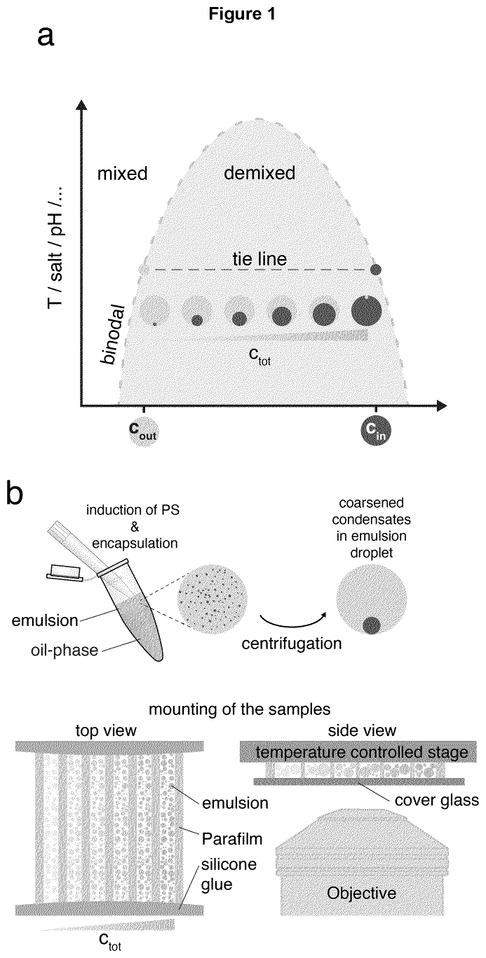

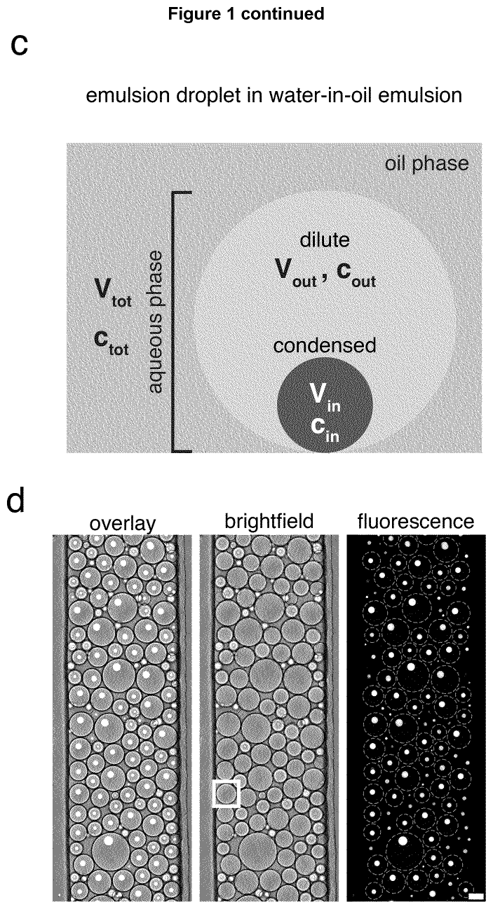

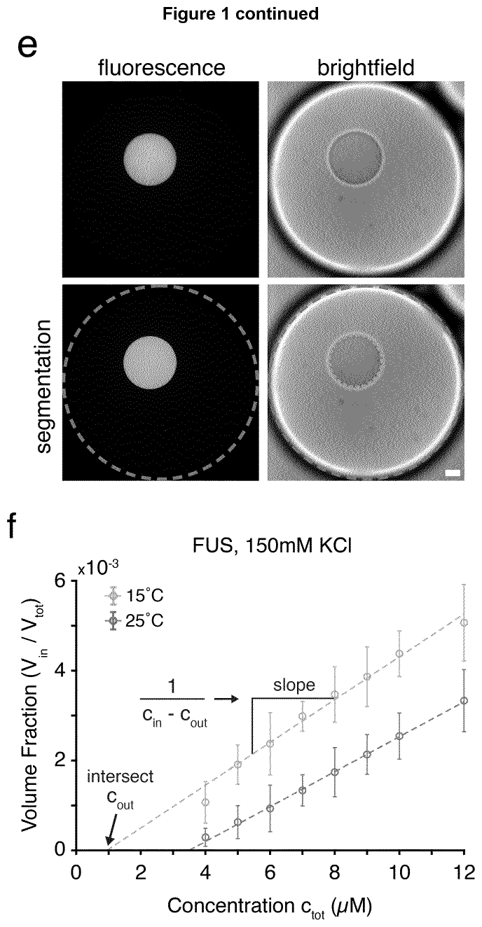

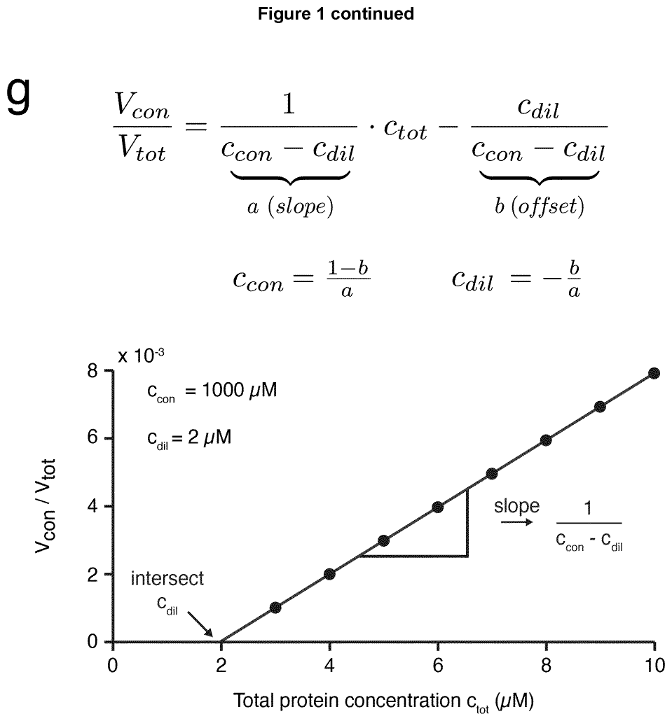

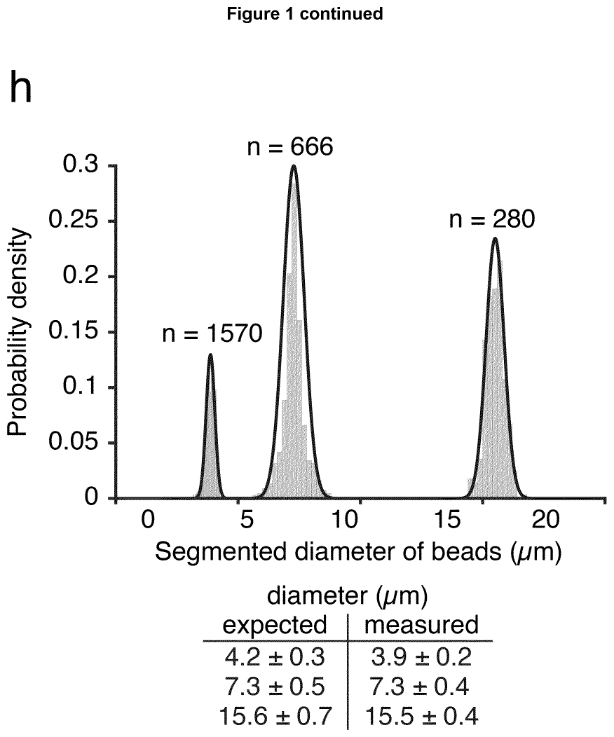

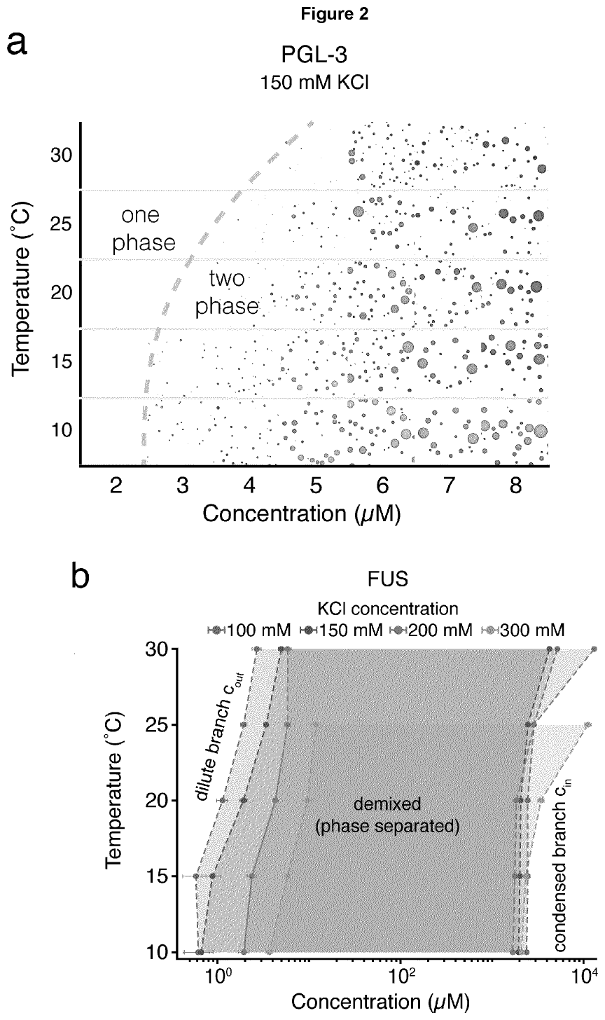

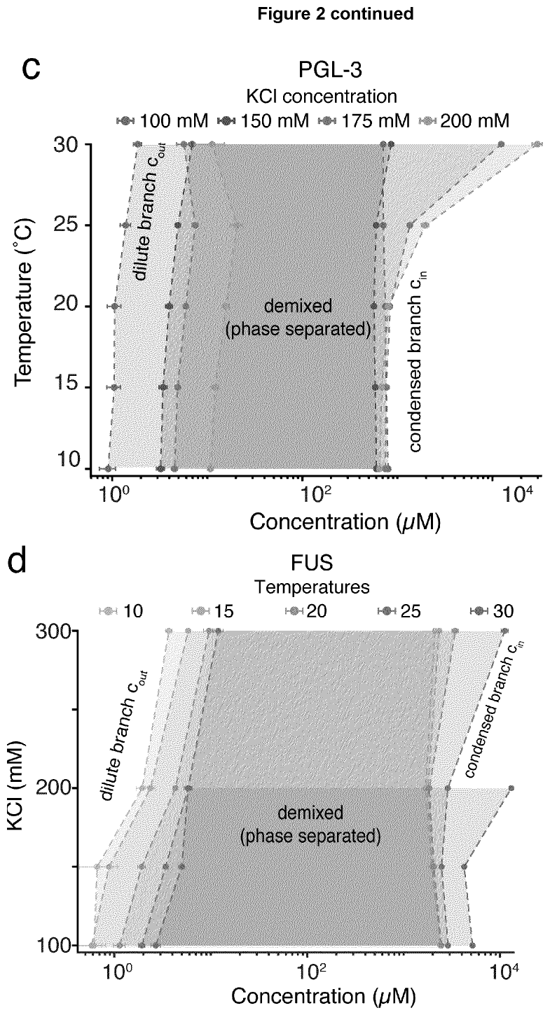

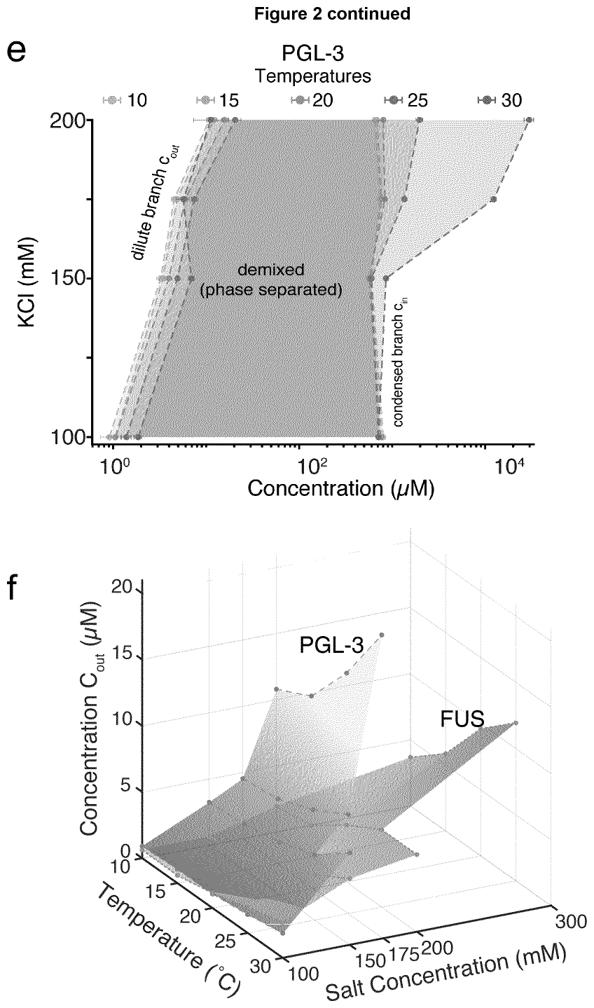

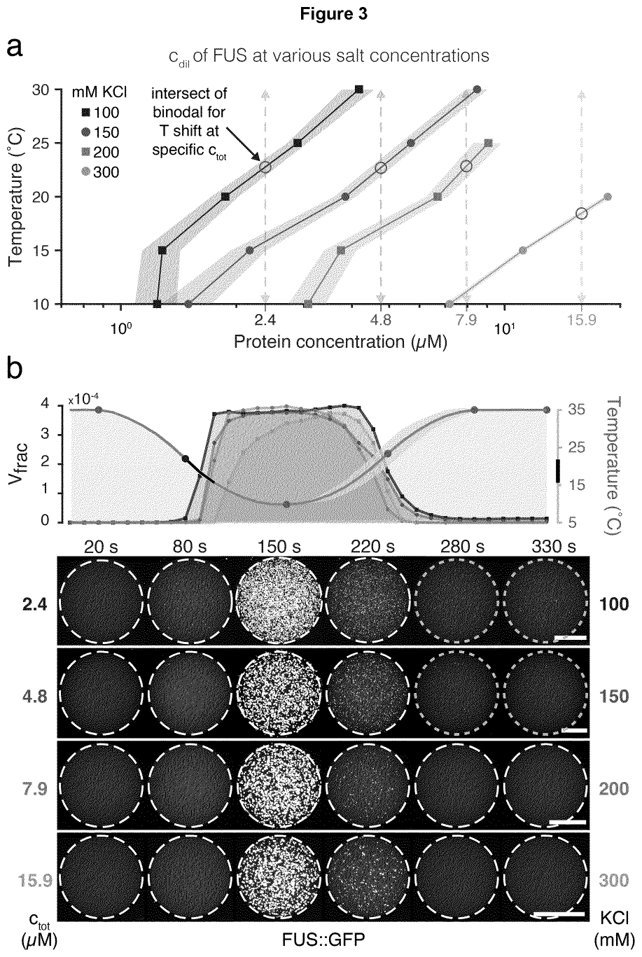

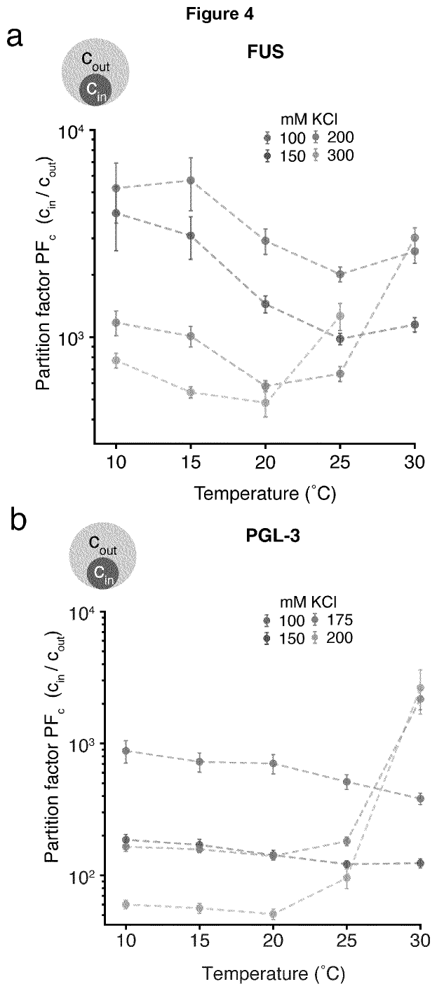

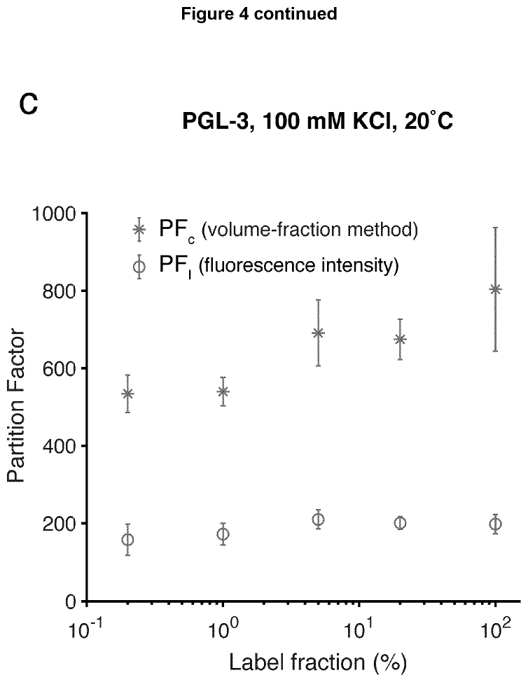

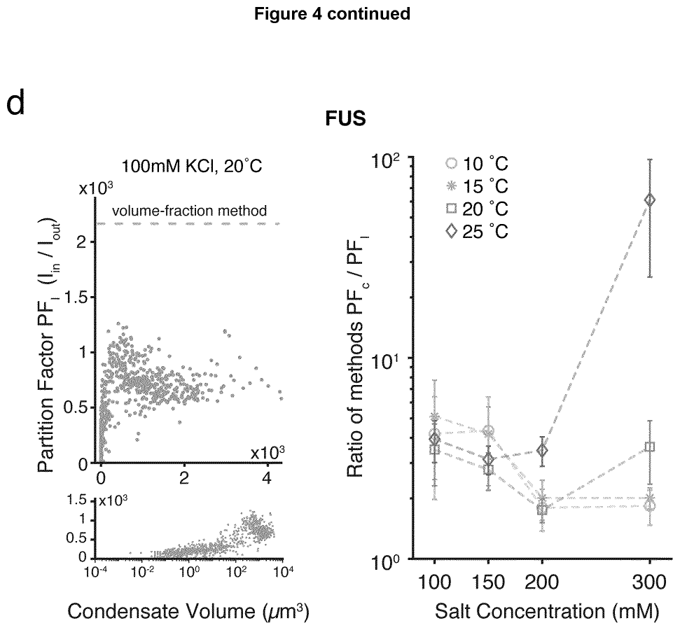

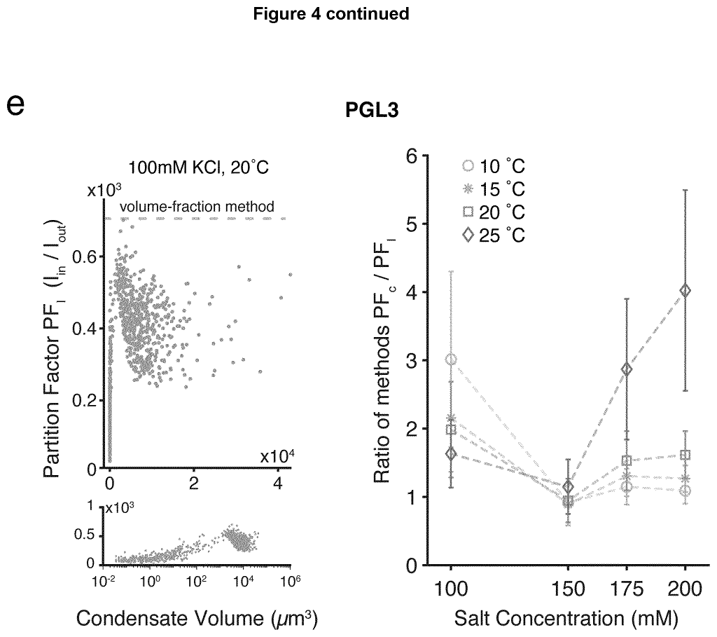

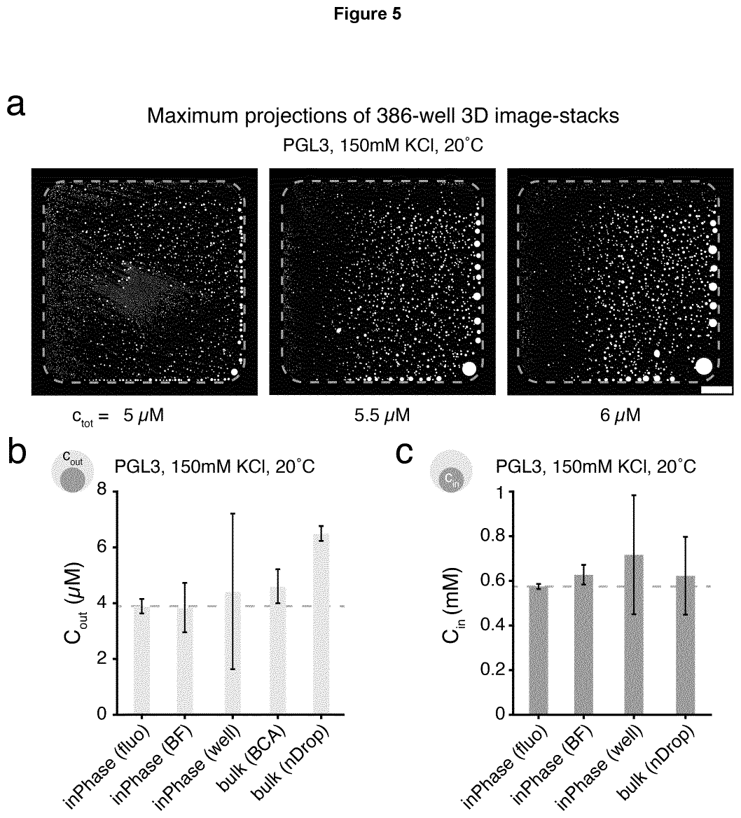

### EXAMPLES

### General Method

The invention relates to the study of protein solutions that undergo phase separation into a dense and a dilute phase. It allows to determine the protein concentrations of both the dense and the dilute phase. It relies on measuring the volume fraction of the condensed phase in the total reaction for a titration series of total protein concentration cₜₒₜ.

$$\begin{matrix}\frac{{V}_{c o n}}{{V}_{t o t}} = \frac{1}{{c}_{con} - {c}_{dil}} \cdot {c}_{tot} - \frac{{c}_{dil}}{{c}_{con} - {c}_{dil}} linear form \\ ( \frac{1}{{c}_{con} - {c}_{dil}} and \frac{{c}_{dil}}{{c}_{con} - {c}_{dil}} are constants )\end{matrix}$$

### Protocol 1 (Water-In-Oil Emulsions):

1. Measure the protein concentration of the stock solutions. The stock solutions can be unlabelled, a mix of labelled and unlabelled or fully labelled protein. Solutions containing only unlabelled protein will be detected using brightfield (or phase-contrast, DIC, etc.) while the mixes that contain labelled protein can be also detected with fluorescence means.
2. Make a titration series of the protein solution.
3. Trigger phase separation of the protein samples. Phase separation can be triggered for example by a change in salt, pH or temperature.
4. Immediately after triggering phase separation, encapsulate in a water-in-oil system (e.g. PicoSurf oil (Sphere Fluidics, UK)).
5. Centrifugation after encapsulation is optional but helps in the segmentation of the condensates. This is because the centrifugation coarsens the condensates.
6. Load the titration series into chambers for microscopy imaging. A custom-built temperature-controlled stage for mounting was used, thereby allowing performing temperature dependent sweep of the binodal.
7. Acquire images of the emulsion droplets and the encapsulated condensates. This allows to determine the volume of the emulsion droplet as well as the condensate. Now the ratio condensate over emulsion droplet volume which is the volume fraction can be determined. In case of fluorescently labelled proteins the determination of the condensate volume can be complemented by fluorescence microscopy.
8. Perform the image and segmentation analysis and fit the measured volume-fractions to a linear regression over the protein concentrations.
9. Determine the c_{con} and c_{dil} using the linear form derived from mass- and volume-conservation equations.
10. Repeat under different conditions (temperature, pH, salt, small molecule concentration) to generate a phase diagram.

### Protocol 2 (Well-Plate):

Step 1.-3. are the same as in Protocol 1.
4. Immediately after triggering phase separation, pipette the samples in the well plate.
5. Centrifugation can be used to coarsen the condensates but in this case, it is preferred to let the condensates sediment for 1 hour.
6. Acquire images of the condensates in the well. The sample volume in a well is known and thus the volume fraction is the result of the segmented condensate volume divided by the sample volume.

Continue with steps 8.-10. in Protocol 1.

### Example 1

### PGL-3 Protein Purification:

PGL-3 was purified from insect cells according to Saha et al. (Cell 166, 1572-1584.e16 (2016)) from SF9-ESF cells were infected with baculovirus containing the PGL-3-GFP-6HIS protein under the polyhedrin promoter. Cells were harvested after 3 days of infection by centrifugation at 500×g for 10 min and then resuspended in lysis buffer (25 mM HEPES 7.25, 300 mM KCl, 10 mM imidazole, 1 mM DTT, 1 protease inhibitor). Cells were lysed by passing the cells 2 times through the LM20 microfluidizer at 15 000 psi. The lysate was then centrifuged at 20 000 rpm for 45 min at 15° C. The lysate was loaded in a pre-equilibrated Ni-NTA column with lysis buffer at 3 mL/min. The Ni-NTA column was rinsed with 10 C.V of wash buffer (25 mM HEPES 7.25, 300 mM KCl, 20 mM imidazole, 1 mM DTT, 1) and the protein was eluted in 1.5 mL fractions with elution buffer (25 mM HEPES 7.25, 300 mM KCl, 250 mM imidazole, 1 mM DTT). After elution the GFP tagged was cleaved to produce untagged PGL-3. The cleavage was performed using a TEV protease overnight at 4° C. PGL-3 and PGL-3-GFP proteins (SEQ ID No: 2) were diluted with Dilution buffer (25 mM Tris pH 8.0, 1 mM DTT) to reach 50 mM KCl before loading the protein in an anion exchange HiTrapQ HP 5 mL column. The HiTrap column was previously equilibrated first with HiTrapQ elution buffer (25 mM Tris pH 8.0, 50 mM KCl, 1 mM DTT) and then with HiTrapQ binding buffer (25 mM Tris pH 8.0, 1 M KCl, 1 mM DTT). The column was mounted in a Akta Pure FPLC system. After the sample was loaded the column was washed with HiTrapQ binding buffer. The sample was finally eluted with a linear gradient from 0 to 55% of HiTrapQ elution buffer (25 mM Tris pH 8.0, 1 M KCl 1 mM DTT) for 25 C.V. Finally, a 100% HiTrap elution buffer step was performed for 5 C.V. The pooled fractions were then loaded in a HiLoad 16/60 Superdex 200 size exclusion chromatography column that was previously equilibrated with Superdex buffer (25 mM HEPES 7.25, 300 mM KCl, 1 mM DTT). After size exclusion, the final samples were collected.

### FUS Protein Purification:

Unlabeled FUS purified from a baculovirus construct containing N-HIS-MBP-FUS-TEV-SNAP. SF9-ESF cells were harvested after three days of infection by centrifugation at 500×g for 10 min. The cell pellet was resuspended using 50 mL of lysis buffer (50 mM Tris pH 7.4, 500 mM KCl, 5% glycerol, 10 mM imidazole, 1 mM PMSF, 1× protease inhibitor) for every 50 mL of cultured cells. The cells were lysed by passing them 2 times through the LM20 microfluidizer at 15 000 psi. The lysate was then centrifuged at 20 000 rpm for 45 min at 15° C. The supernatant was collected and loaded into a Ni-NTA column that was previously equilibrated with lysis buffer. After loading the sample, the column was washed for 10 C.V. with Ni-NTA was buffer (50 mM Tris pH 7.4, 500 mM KCl, 5% glycerol, 20 mM imidazole). The protein was then eluted with Ni-NTA elution buffer (50 mM Tris pH 7.4, 500 mM KCl, 5% glycerol, 300 mM imidazole). The collected fractions where then loaded into a MBPTrap HP column preequilibrated with Ni-NTA elution buffer. The MBP column was washed for 10 C.V with MBP wash buffer (50 mM Tris pH 7.4, 500 mM KCl, 5% glycerol). After washing, the sample was eluted with MBP elution buffer (50 mM Tris pH 7.4, 500 mM KCl, 5% glycerol, 500 mM arginine, 20 mM maltose). The protein was diluted to a concentration of less than 15 μM using MBP elution buffer. 3C and TEV proteases were then added to cleave the MBP and SNAP tags from the FUS construct. The cleavage reactions were incubated overnight at 18° C. Finally, the protein was loaded in a SepFast GF-HS-L 26×600 mm gel filtration to remove the cleaved MBP and SNAP tags and exchange the buffer. The SepFast column was previously equilibrated in storage buffer (50 mM HEPES pH 7.25, 750 mM KCl, 5% glycerol, 1 mM DTT). The sample was concentrated to a final concentration of ˜15 μL using 30 kDa Amicon centrifuge filters. FUS-GFP (SEQ ID No: 1) was purified as previously described (Wang, J. et al. “A Molecular Grammar Governing the Driving Forces for Phase Separation of Prion-like RNA Binding Proteins”, Cell, 2018, 174, 688-699.e16).

### Protocol In Vitro Protein Handling:

Protein solutions were prepared from stocks that are frozen at −80° C. The protein stock solutions were cleared from potential aggregates using centrifugal filters (UFC30HV00, Merck, Germany). The protein concentrations were determined via extinction coefficient measurements at 280 nm (NanoDrop, Thermo Fisher Scientific, USA). Next, the desired label fraction, in the main experiments 5% GFP labeled protein, was prepared. In order to keep the protein from phase separating to early the titration of the final protein concentrations was done at salt concentrations of the protein stock (300 mM KCl for PGL-3 and 750 mM KCl for FUS; both 25 mM HEPES and 1 mM DTT at 7.4 pH). Only in the final step the salt concentration was dropped to the desired value, using a no salt buffer (25 mM HEPES and 1 mM DTT at 7.4 pH), and the solution was immediately encapsulated into the water-in-oil solution. Encapsulation was performed using twice the amount of PicoSurf oil (2% (w/w) in Novec 7500, Sphere Fluidics, UK) compared to the protein solution (usually 5 μL). After adding the oil on top of the protein solution in standard 100 μl microcentrifuge tubes the solution is agitated using a 10 μl pipette until the desired size distribution of water-in-oil solutions is reached. This solution can be used directly for imaging. Alternatively, the coarsening of the phase separated condensates can be speed up by a mild centrifugation step of the water-in-oil emulsions (100-1000 g for 3 min). This will lead to predominantly one single condensate of condensed phase per emulsion droplet.

### Preparation of Emulsions:

Protein solutions were prepared from stocks that are frozen at −80° C. The protein stock solutions were cleared from potential aggregates using small volume centrifugal filters (UFC30W25, Merck, Germany). The protein concentrations were determined via extinction coefficient measurements at 280 nm (NanoDrop, Thermo Fisher Scientific, USA). Next, the desired fraction of labelled protein was prepared (main experiments 5% GFP labelled protein for PGL-3, 100% for FUS). The protein concentrations were adjusted using the same salt concentration as the protein stock (300 mM KCl and 25 mM HEPES for PGL-3 and 500 mM KCl and 50 mM HEPES for FUS; both 1 mM DTT at pH 7.4). In the final step the salt concentration was reduced to the desired value, using a buffer containing no salt (HEPES and 1 mM DTT at pH 7.4) to trigger phase separation. The protein solution was then immediately encapsulated into a water-in-oil solution. Encapsulation was performed using twice the amount of PicoSurf oil (2% (w/w) in Novec 7500, Sphere Fluidics, UK) compared to the protein solution. Here, we add the oil to the protein solution in standard 100 μl PCR tubes and the solution is agitated using a 20 μl pipette and 200 μl tips until the desired size distribution of water-in-oil solutions is reached. To coarsen the phase separated condensates within the emulsion drops we used a centrifugation step (200-400 g for 4 min) at the desired temperature of the first data point. This will lead to a single condensate per emulsion droplet in most emulsion droplets. A titration series of the total protein concentration was then mounted on a temperature controlled microscope stage.

### Sample Loading and Imaging Procedure for Emulsions:

As a sample holder a custom-made temperature stage based on Peltier elements connected to a sapphire microscope slide (25×75 mm) was used. This allows for fast and precise temperature changes both below and above room temperature. Between the sapphire slide and a standard cover slide (22×22 mm, 170 μm, Menzel, Germany) lanes of Paraflim (Bernis, USA) stripes were sandwiched. The sandwich is connected by heating the stage to at least 55° C. which will connect the Parafilm to the top and bottom by applying soft pressure on the cover slide. This allows to load multiple samples in a separated fashion in one measurement. Emulsion solutions are loaded into the individual lanes by pipetting the turbid upper layer in the microcentrifuge tubes. Each lane is sealed using addition curing silicone (Picodent twinsil speed, Picodent, Germany) to ensure no evaporation and movement of the water-in-oil emulsions upon temperature changes. Sample imaging was performed via CellSens software (Olympus, Japan) on an Olympus IX83 microscope connected to a Yokogawa W1 SoRa spinning-disc system (Yokogawa, Japan) and a Hamamatsu Orca Flash v3 sCMOS camera (Hamamatsu, Japan) using a 40× air objective (UPLXAPO, 0.95NA, Olympus, Japan). For each sample, parafilm lane, a multi-tile brightfield image and a corresponding multi-tile 3-dimensional fluorescence stack was recorded using a piezo stage (Mad city labs, USA). The main temperature experiments started at 10° C. and increased the temperature by 5° C. steps. Between consecutive temperatures we added 5 min of equilibration time before imaging.

### Image Analysis:

Images were processed by a custom-written pipeline using MATLAB software (The Mathworks, USA). The brightfield images of each lane were used to detect the contours of the water-in-oil emulsion droplets. The location as well as the volume of each emulsion droplet was determined considering the height of approximately 100 μm of the Parafilm chambers. Corresponding positions in the fluorescence image stack are further analyzed after removing emulsion droplets that are intersected by the margins of the imaged tiles as well as overlapping emulsions. For each emulsion droplet its condensed phase is determined by image registration and parameters like total condensed phase and intensity values are extracted. In brief, after basic image filtering, an intensity gradient approach was used to find a good estimate for the outline of the condensates.

The inventors then assume sphericity of each condensate to calculate its volume. For large condensates the spherical assumption might introduce overestimation of the actual volume since they can flatten out due to gravity. To get valid estimates for the dilute phase intensities the inventors first dilate the masks of the condensed phase and remove them from the mask of the emulsion droplet. This helps to minimize the influence of intensity values close to the margins of the condensate phase. Furthermore, the inventors can also use the brightfield images to estimate the volume of the condensed phase. The image analysis pipeline was calibrated using monodisperse fluorescent beads of known size immersed in glycerol solution to mimic the refractive index difference found for condensates in buffer.

### Calculation of Dilute and Condensed Branch Concentrations:

To get to the form of Equation 1 the inventors assume volume and mass conservation in the reaction container. While there could be processes that lead to a change of total volume in case of a phase transition in our case of protein phase separation the actual volumes of the condensed phase are orders of magnitude smaller than the total reaction volume and thus even in such a case the linear form will be valid for small volume fractions V_{frac}=V_{con}/Vₜₒₜ.

The conservation reads as Vₜₒₜ=V_{dil}+V_{con} for the volume and as Vₜₒₜ·cₜₒₜ=V_{dil}·c_{dil}+V_{con}·c_{con} for the mass given that the mass equals the volume times the concentration (m=V·c). Converting the mass conservation to

$${V}_{frac} = \frac{{c}_{tot}}{{c}_{con}} + \frac{{V}_{dil}}{{V}_{tot}} \cdot \frac{{c}_{dil}}{{c}_{con}}$$

and substituting the volume conservation as

$$\frac{{V}_{dil}}{{V}_{tot}} = 1 + \frac{{V}_{con}}{{V}_{tot}}$$

leads to

$${V}_{frac} = \frac{1}{{c}_{con} - {c}_{dil}} \cdot {c}_{tot} - \frac{{c}_{dil}}{{c}_{con} - {c}_{dil}} . \qquad ( Equation 1 )$$

The volume fraction data for each total concentration was pooled from all repeats and used for linear regression of the concentration series in cₜₒₜ. Using the linear form V_{frac}=a·cₜₒₜ+b the inventors get

$${c}_{con} = \frac{1 - a}{b} and {c}_{dil} = - \frac{b}{a}$$

according to Equation 1. Via the confidence interval of the fit and error propagation, the inventors can determine an error for the dilute- and condensed-phase concentrations.

### Example 2. Time Dependent Behavior of the Condensates

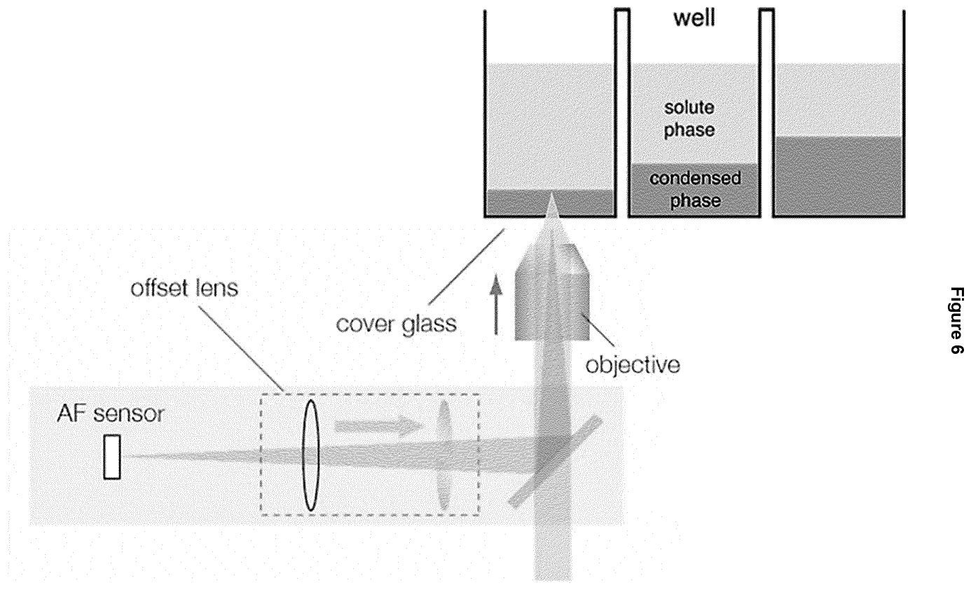

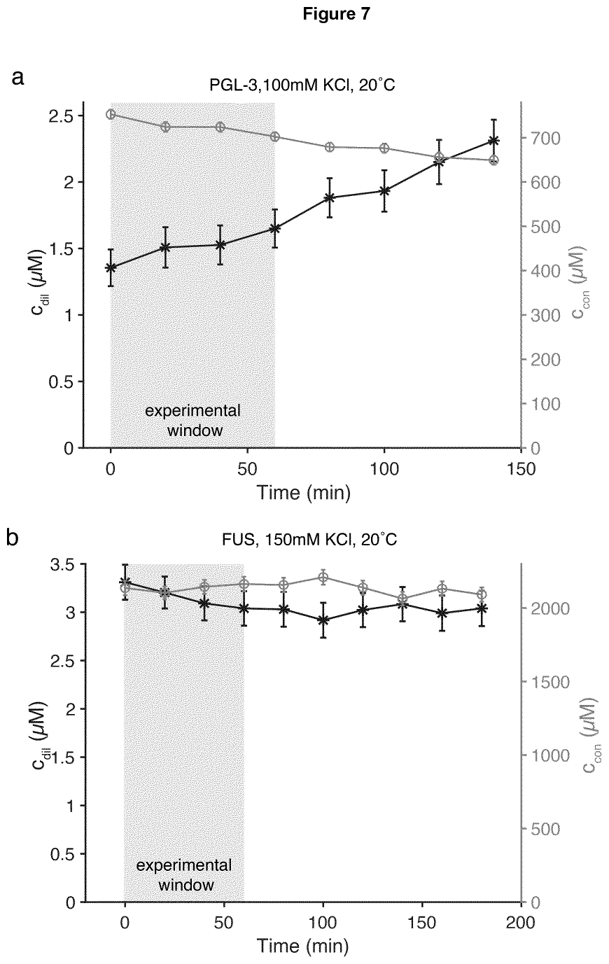

It has been observed, that the material properties of condensates change over time. The material properties of a condensate are relevant for their function. This is especially interesting for the ageing of condensates that are linked to neurodegenerative diseases (e.g. hardening of FUS condensates related to ALS). However, it remains challenging to measure material properties on such small scales. Here, it is shown that changes in the phase (diagram) can be tracked over time using the inventive approach (FIG. 7).
1. Prepare the sample according to protocol 1 or 2 (e.g., a total protein concentration titration at phase separating conditions, at the desired external conditions (temperature, salt concentration, pH, pressure, buffer composition, crowding agent)).
2. Perform time-dependent measurement of the volume fraction of the condensed phase (e.g. via fluorescence microscopy based imaging using a labeled protein, or brightfield imaging using unlabeled protein, or any of the other techniques outlined herein).
3. Use linear regression to fit the volume fraction versus total concentrations data for each time-point and apply our formula to get to a time-dependent cᵢₙ(t) and cₒᵤₜ(t).
4. A time-dependent plot of either cᵢₙ or cₒᵤₜ (or any derivative value of the two, e.g. the ratio) allows assessing the ageing of the protein condensed phase of interest.

### Example 3. Effect of Small Molecule Drugs

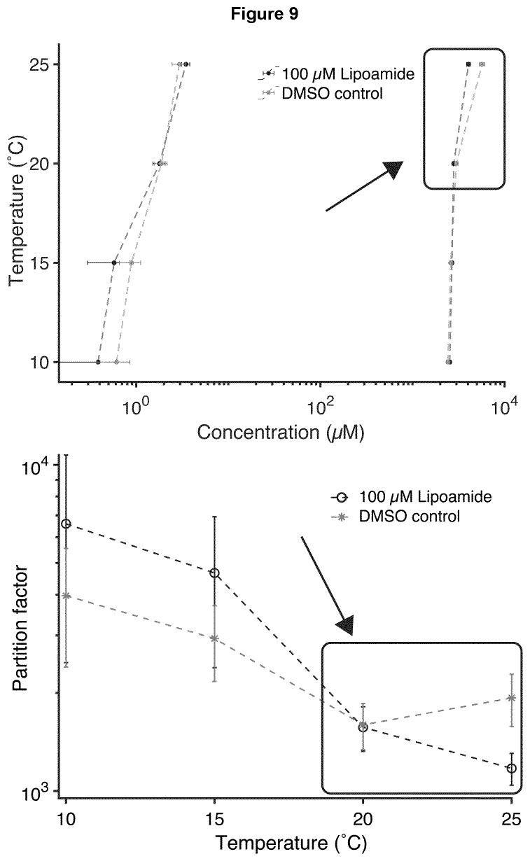

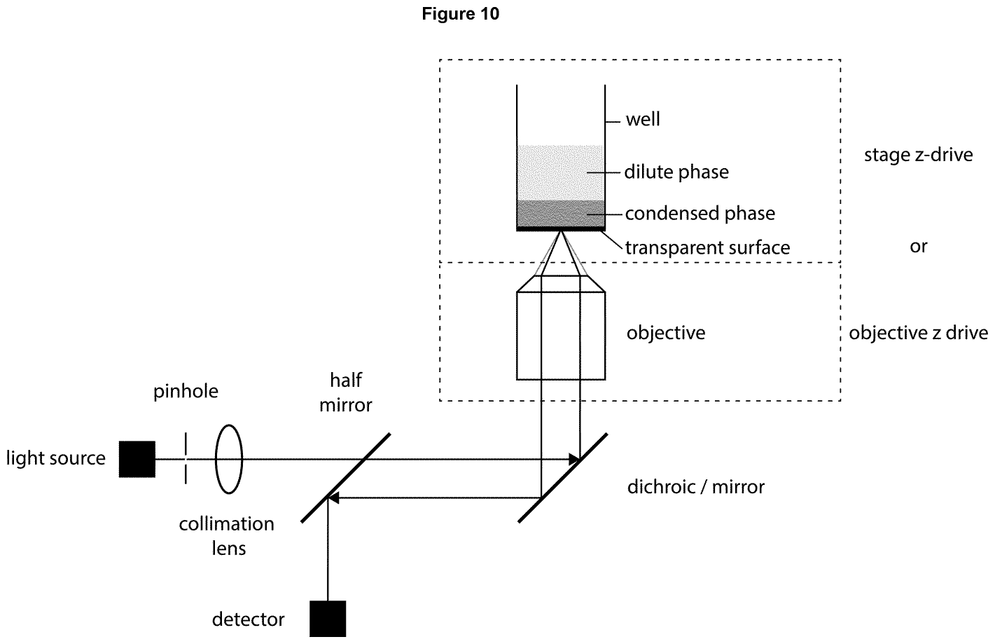

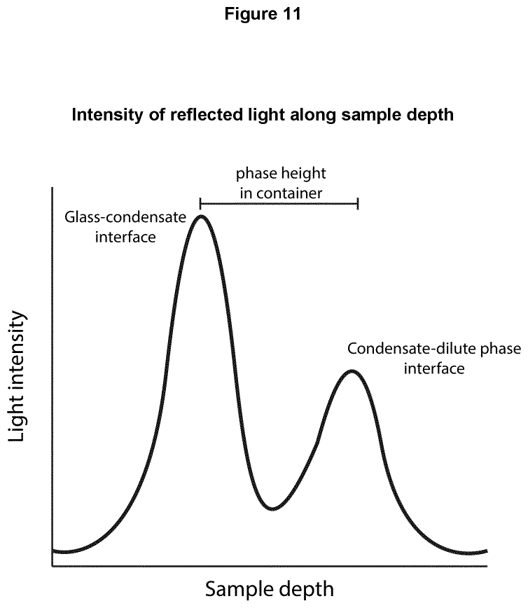

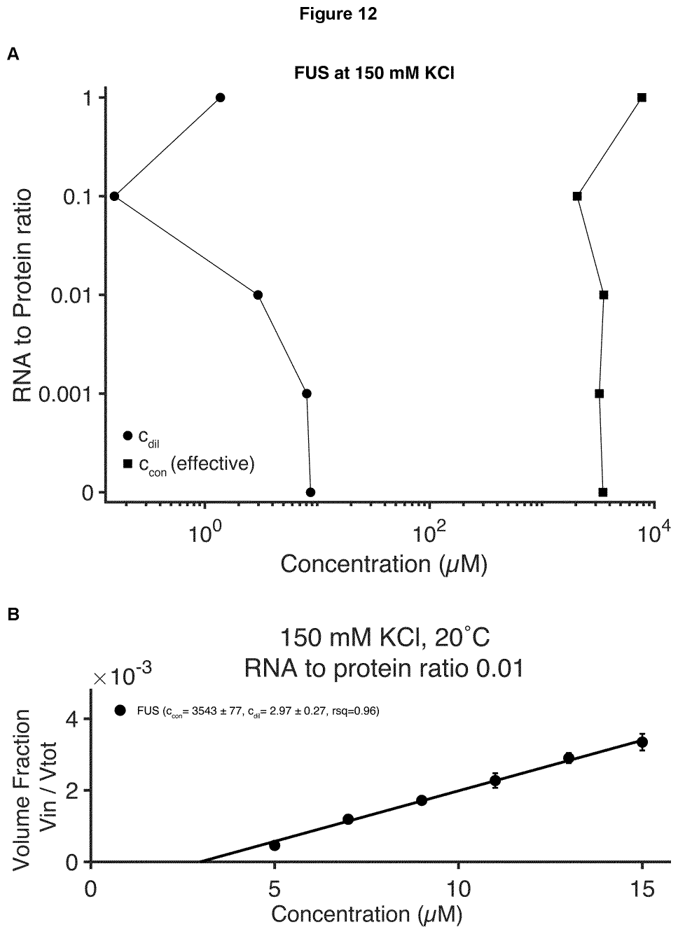

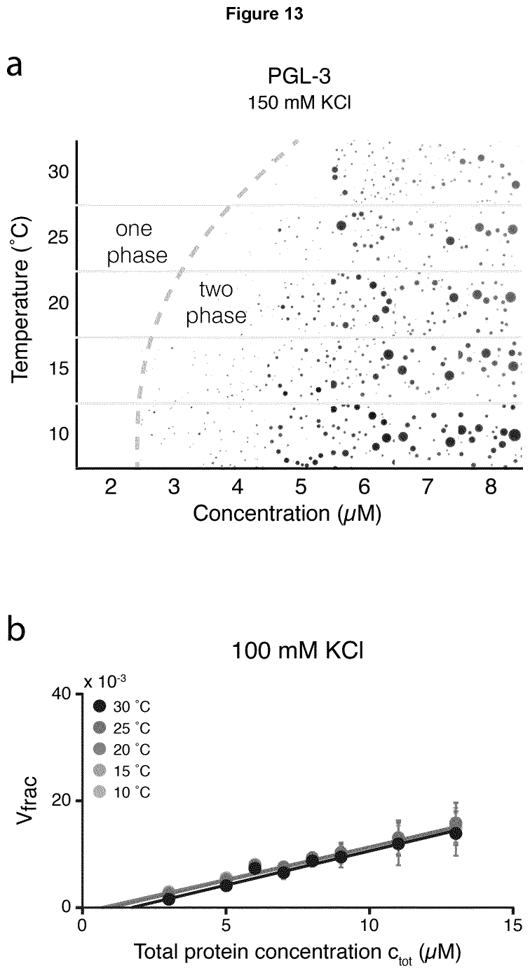

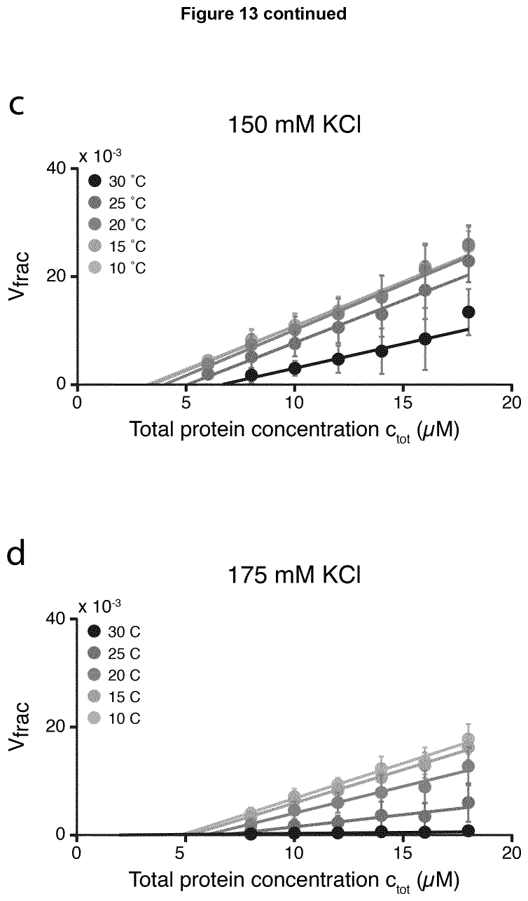

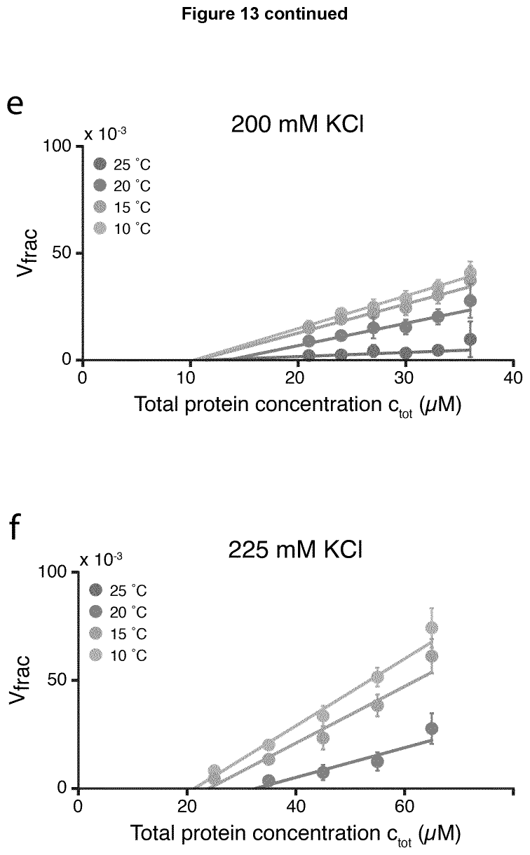

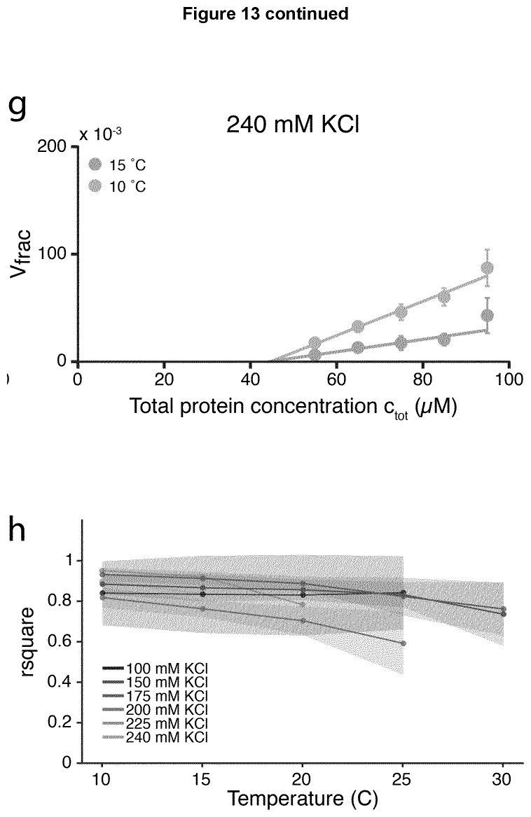

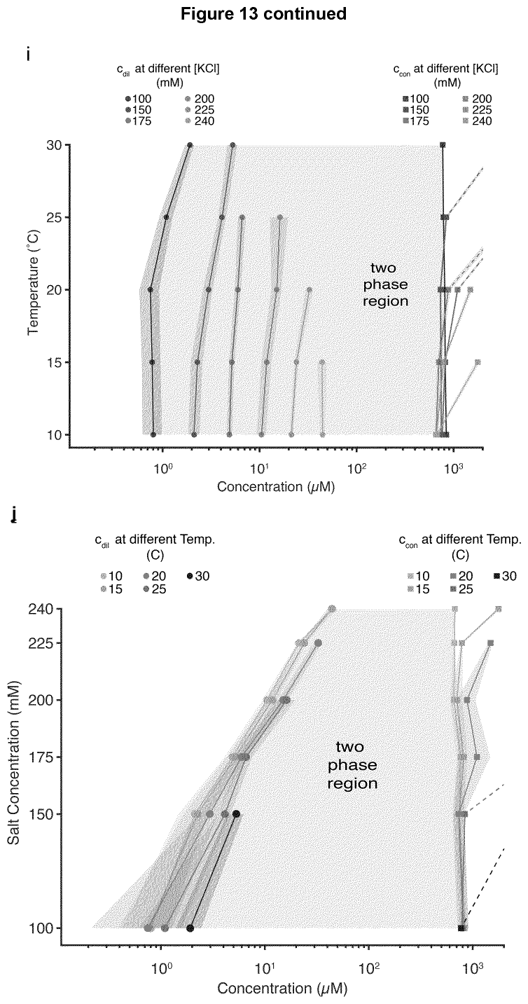

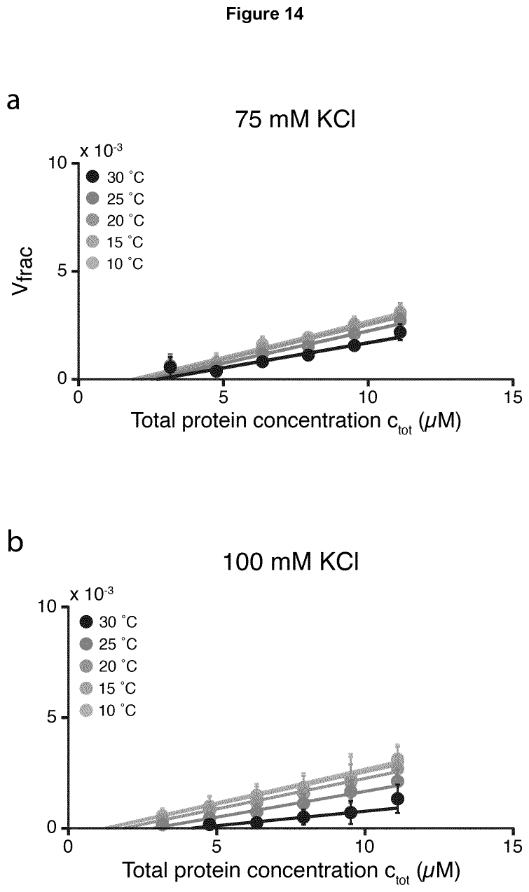

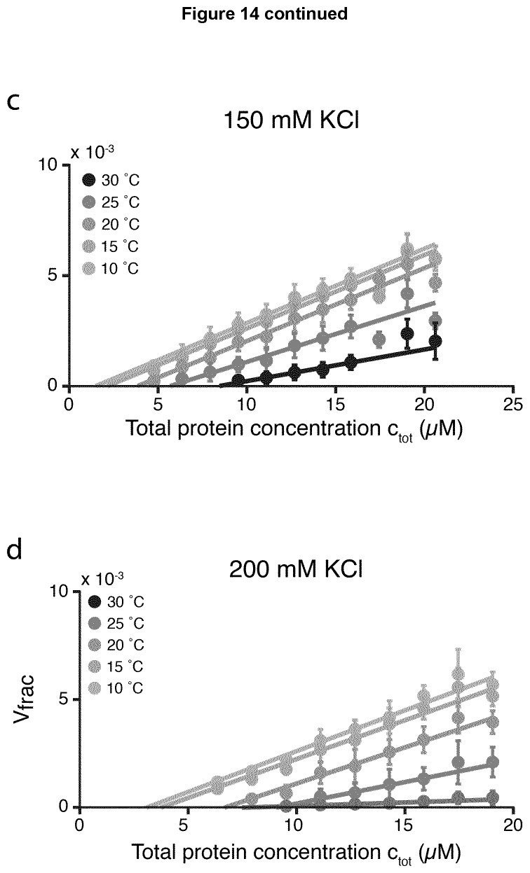

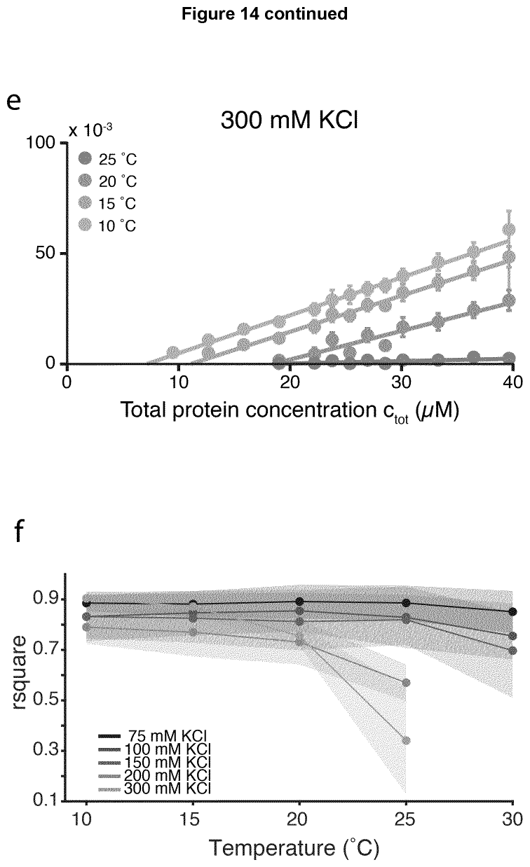

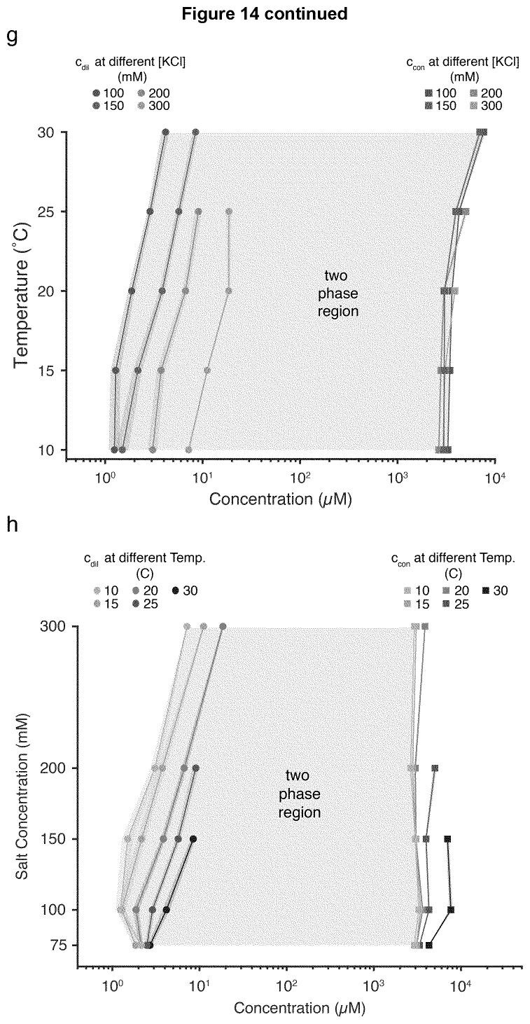

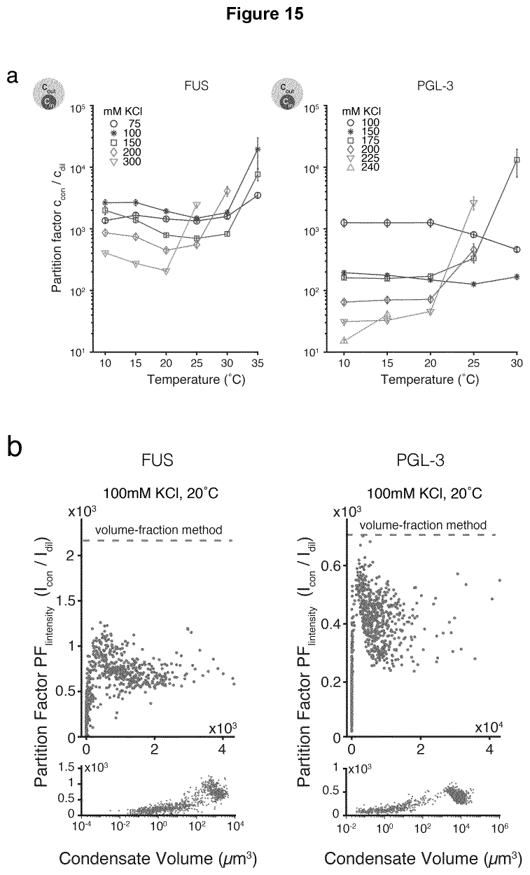

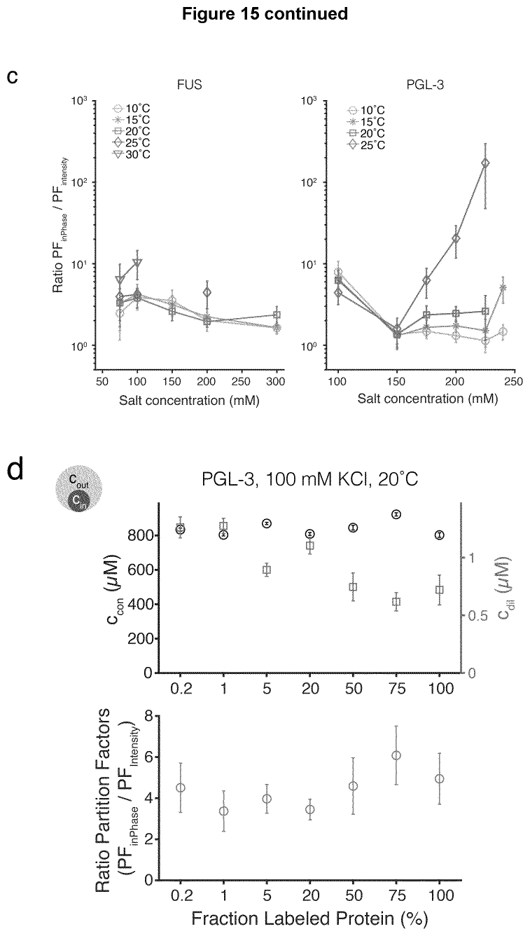

To demonstrate the possibilities of drug screening, a small molecule that has been indicated to affect the phase separation of FUS, a protein related to ALS has been used. It has so far been difficult to accurately the extent of the effect because of a lack of quantitative measures (FIG. 9).

Here the effect of Lipoamide on the phase behavior of FUS is shown, where a reduced amount of conformational changes at higher temperatures was observed.

This is indicated by the earlier bent of the condensed phase branch of the binodal for the untreated control (DMSO). This can also be seen in the partition factor cᵢₙ/cₒᵤₜ.
1. Prepare the sample according to protocol 1 or 2 (e.g., a total protein concentration titration at phase separating conditions).
2. Add small molecule drug of interest to the final protein mix before encapsulation for the emulsion approach or before/after adding protein mix to the well-plates (depending on intention to measure the effect of the drug during formation of the condensates (before) or on the already formed condensates (after)).
3. Use linear regression to fit the volume fraction versus total concentrations data for each drug concentration and apply our formula to get to a dose-dependent cᵢₙ and cₒᵤₜ compared to a control (e.g., DMSO control).
4. This allows preparing an IC50 plot for the effect of the small molecule drug.

### Example 4. Phase Diagrams In Vivo

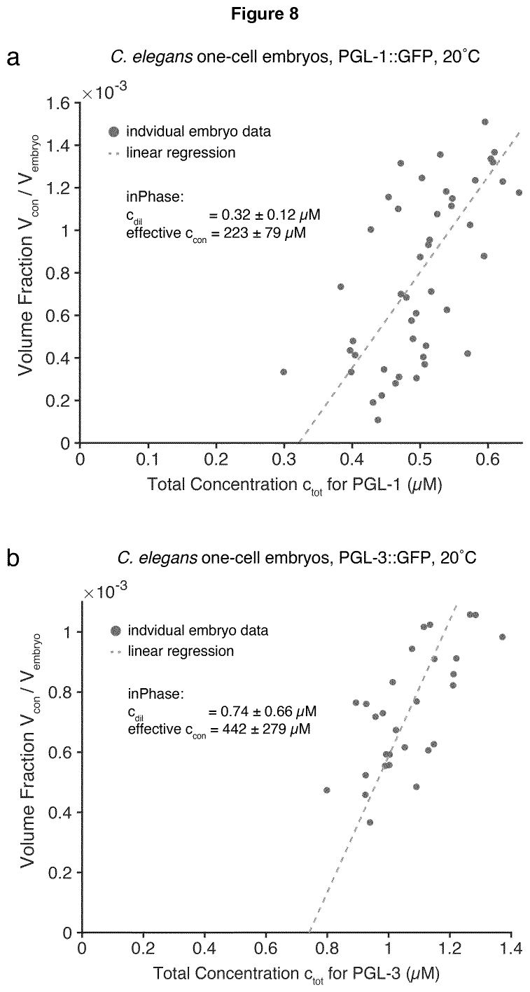

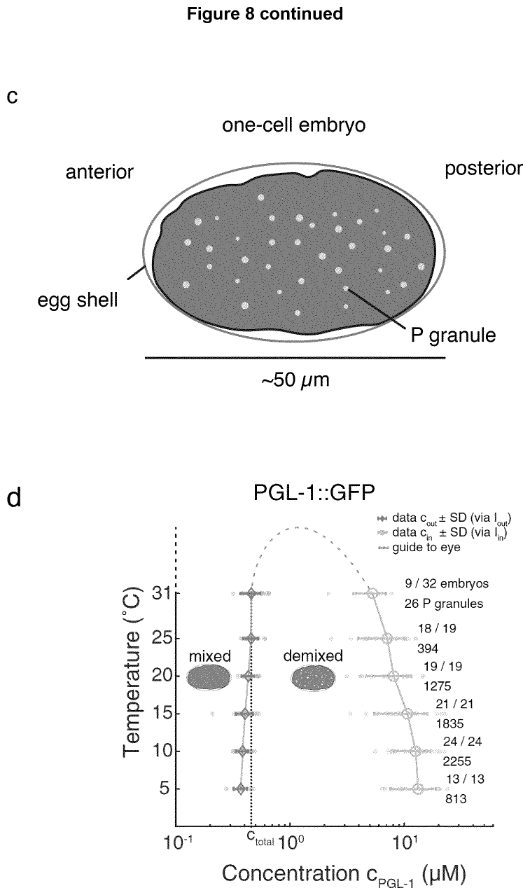

In general, the method of the present invention can be used for any phase separating system. This is also true for in vivo settings of phase separating proteins. The natural or induced fluctuations in the total protein concentration of cells are equivalent to our protein concentration titration series. Thus, the inventive method can be applied to calculate c_{dil} and c_{con}. As previously discussed, the volume fraction of the phase and the total concentration of protein (e.g. via GFP labelled protein) is determined. This is shown exemplary for C. elegans embryos and the proteins PGL-1 and PGL-3 that are constitutive for P granules (FIG. 8).
1. Determine the volume fraction of the condensed phase of a protein inside a cell (e.g., using a fluorescently labelled protein (live imaging) or fixed cells with immunofluorescence).
2. Determine the total concentration of the protein of interest within the given cell. For example, by using the mean intensity of the protein in the cell and relating it to a known standard (e.g., fluorophore concentration titration, quantitative immunoblots see (Fritsch et al. PNAS, 2021, 118(37), e2102772118), mass spectrometry see (Saha et al Cell 166, 1572-1584.e16 (2016))).
3. Use the intrinsic variability of the total concentration between cells to plot the volume fraction over total protein concentration.
4.a. In addition, RNAi can be used to decrease the total concentration of a given protein in vivo in a controlled way.
4.b In addition, inducible gene expression (e.g. doxycycline based) can increase the total concentration of a given protein in vivo in a controlled way. The gene can be integrated in the genome or transfected in a plasmid.
5. Use linear regression to fit the volume fraction versus total concentrations data for and apply equation 1 to get c_{dil} and an apparent c_{con}.

### Example 5: Effect of RNA on Protein Phase Separation

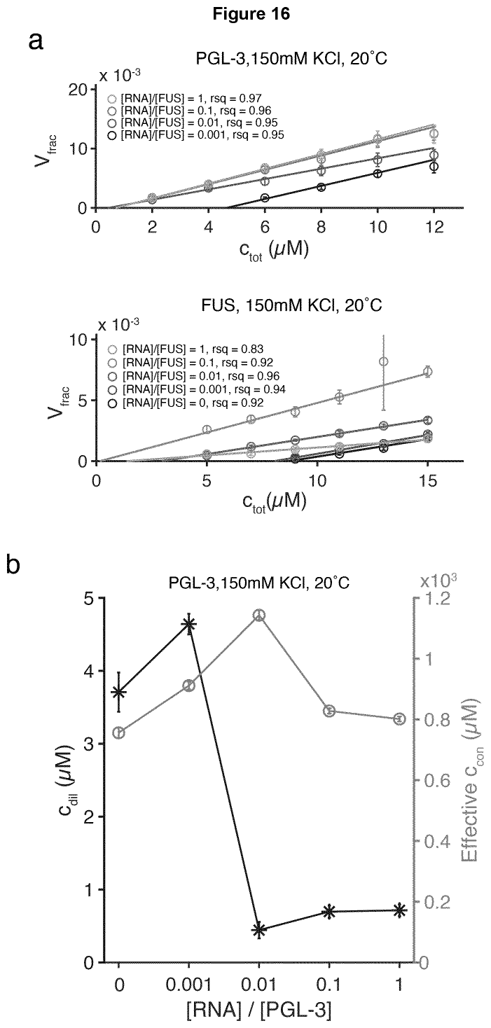

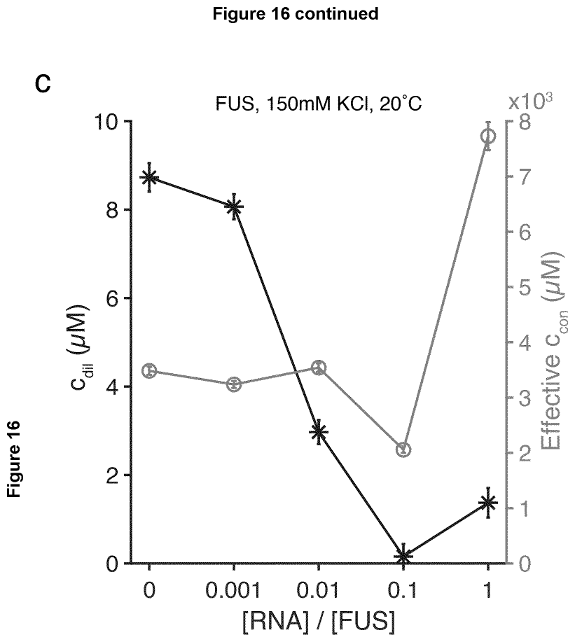

FUS-GFP containing samples were prepared as described in Example 1. Phase separation was triggered by simultaneously lowering the salt concentration of the sample and adding the Poly(A) RNA in an amount to maintain a fixed molar ratio of Poly(A):FUS-GFP. The volume fraction of the condensed phase was determined as described in Example 1 and c_{dil} and c_{con}, were determined, wherein c_{con} represents an apparent concentration containing both Poly(A) (SEQ ID No: 3) and FUS-GFP concentration. The concentration of the single components of the sample can be resolved by the knowledge of the molar ratio of RNA to protein in the condensed phase (FIG. 16).

### Example 6: Multi-Well Plate Assay

The inventors load equal amounts of 20 μl sample volume for the total protein concentration titration in 384 well plates (PhenoPlate, PerkinElmer, USA). The well plates were chosen due to the superior non adhesive properties that allow to assume spherical shape of the condensates. Well plates are centrifuged at 200 g for 10 min with low acceleration and deceleration of the rotor to minimize coarsening into one condensate. The segmentation routine used for the emulsion-based approach was calibrated for the well plates and used to determine the volume fraction of the condensed phase for each total concentration. The inventors then use equation 1 to derive c_{dil} and c_{con} and the respective errors.

### Example 7: Bulk Concentration Measurements

Centrifugation of a phase separated suspension provides us with the supernatant containing the dilute phase and a condensed phase at the bottom of a PCR tube that can both be used for the bulk measurements when using large amounts of sample. The inventors could use measured volume fractions of condensed phase for given external parameters to arrive at volumes of condensed phase suitable for pipetting. The inventor used the Pierce BCA protein assay kit (ThermoFischer, USA) together with a TECAN Spark 20M plate reader (TECAN, Swiss) as measurement a triplicate of an BSA standard curve was used to convert intensities to concentration. For each measurement a triplicate of a total concentration (cₜₒₜ) series below and above the saturation concentration (c_{dil}) were carried out. Below c_{dil} we see a match of cₜₒₜ and the measured concentrations (c_{BCA}) and as expected above c_{dil} a plateau-like region for c_{BCA}. The intersect of linear fits to the plateau and the initial slope provides us with c_{dil}. To determine the condensed phase concentration c_{con} via fluorescence absorption measurement with a NanoDrop spectrometer (ThermoFisher, USA) the inventors used a negative displacement pipette (Eppendorf, Germany). This was necessary because of the high viscosity of the condensed phase and potential errors using air pipettes. A known volume of condensed phase was diluted into high salt buffer before absorption measurement to ensure dissolution of the phase. In addition, guanidinium chloride can be used to dissolve condensates for proteins that would not dissolve again in high salt buffer.

### Example 8: Temperature Shift Experiments

Emulsions were prepared as described in the section “Preparation of emulsions” as described in Example 1 except without a final centrifugation step. This step was not necessary since the total protein concentrations were adjusted to be outside the binodal at the temperatures used for preparation. We used FUS::GFP at the salt concentrations of 100, 150, 200, and 300 mM KCl at protein concentrations of 2.4, 4.8, 7.9, and 15.9 μM respectively (all pH 7.4). A low density of emulsion droplets was mounted in Parafilm chambers on the temperature-controlled stage to ensure stationary emulsions during temperature shift experiments. Images were recorded using the described spinning disc confocal system at 10 s temporal resolution and 1 μm resolution in the z-axis using a 40× air objective.

### Example 9: One-Cell C. elegans Embryo Experiments

One-cell embryos were imaged and analyzed according to a protocol described previously (Fritsch, A. W. et al. Local thermodynamics govern formation and dissolution of Caenorhabditis elegans P granule condensates. PNAS, 2021, 118.). This allowed to derive the volume fraction of condensed phase as well as the total concentration of labelled protein per embryo. In brief, known total protein concentrations (PGL-1::GFP, PGL-3::GFP respectively) in the different worm strains were related to fluorescence intensities to derive a scaling factor between florescence intensity and concentration. To derive the total condensate volume as well as the embryo volume, the inventors used a custom-written MATLAB (The MathWorks) segmentation routine. The worm lines used were TH586, pgl-1::mEGFP pgl-1(dd54[pgl-1::mEGFP]) and TH561, pgl-3::mEGFP (pgl-3(dd29[pgl-3::mEGFP]). Here, the proteins PGL-1 and PGL-3 were labelled at their endogenous genomic locus with monomeric enhanced GFP using the co-CRISPR method.

1. A method for determining a concentration (c_{con}) of a polymer in a condensed phase and a concentration (c_{dil}) of the polymer in a dilute phase in a system comprising:
A) determining a total volume (Vₜₒₜ) of the system and a total concentration (cₜₒₜ) of the polymer;
B) triggering phase separation of the polymer into a condensed phase and a dilute phase in said system;
C) determining a volume fraction (V_{con}/Vₜₒₜ) of the condensed phase of the polymer in said system;
D) determining a concentration (c_{con}) of the condensed phase of the polymer and a concentration (c_{dil}) of the dilute phase of the polymer in said system by using a following linear form equation:
$$\frac{{V}_{c o n}}{{V}_{t o t}} = \frac{1}{{c}_{con} - {c}_{dil}} \cdot {c}_{tot} - \frac{{c}_{dil}}{{c}_{con} - {c}_{dil}} ,$$
E) changing at least one condition of said system and repeating the steps A) to D).

2. The method according to claim 1, wherein the polymer is a protein, or a polynucleic acid, preferably RNA, DNA, a mixture of a protein and RNA or a mixture of a protein and DNA and/or wherein the system is an in vitro system and is selected from a solution, an emulsion, or cells.

3. The method according to claim 1, wherein in step B), the phase separation of the polymer in said system is triggered by changing: the concentration of a component in the system selected from a salt, a crowding agent, or a buffer; the pH value; the pressure; or the temperature of the system.

4. The method according to claim 1, wherein when the polymer is not labelled with a fluorophore or a fluorescent protein, in step A) the total volume (Vₜₒₜ) of said system and the total concentration (cₜₒₜ) of the polymer are determined by means of bright-field, dark-field, phase-contrast, holographic, polarization, or differential interference correlation (DIC) microscopy, or light-scattering based approaches.

5. The method according to claim 1, wherein when the polymer is labelled with a fluorophore or a fluorescent protein, in the step A)
the total volume (Vₜₒₜ) of said system and the total concentration (cₜₒₜ) of the polymer are determined by means of fluorescence microscopy.

6. The method according to claim 1, wherein step C) comprises:
C1a) encapsulating a portion of the system in an emulsion system;
C2a) determining a volume (Vₑₘ) of one emulsion droplet of the emulsion system, and a condensed volume (V_{con}′) of the condensed phase of the polymer in said emulsion system;
C3a) determining a volume fraction (V_{con}′/Vₑₘ) of the condensed volume (V_{con}′) of the condensed phase of the polymer in said emulsion system and the volume (Vₑₘ) of one emulsion droplet of said emulsion system, wherein the measured volume fraction (V_{con′}/Vₑₘ) is identical to the volume fraction (V_{con′}/Vₜₒₜ) of the condensed volume (V_{con}) of the condensed phase of the polymer in the system and the total volume (Vₜₒₜ) of the system.

7. The method according to claim 6, wherein in the step C2a)
a volume (Vₑₘ) of the emulsion system, and a condensed volume (V_{con}′) of the condensed phase of the polymer in said emulsion system are determined by means of fluorescence microscopy.

8. The method according to claim 6, further comprising after the step C2a):
C2b) adjusting the measured fluorescent image with a predetermined partition factor.

9. The method according to claim 1, further comprising after step B):
B′) centrifuging the system.

10. The method according to claim 1, wherein in step E)
the at least one condition of said system is selected from a concentration of a component in the system wherein the component is selected from a salt, or a buffer;
the pH value; the pressure; a crowding agent; or the temperature of the system, or a combination of the aforementioned conditions.

11. An assay method for identifying bioactive compound(s), comprising:
A) determining a total volume (Vₜₒₜ) of the system and a total concentration (cₜₒₜ) of a polymer in the system;
A′) adding a predetermined amount of test compounds into said system;
B) triggering phase separation of the polymer into a condensed phase and a dilute phase;
C) determining a volume fraction (V_{con}/Vₜₒₜ) of the condensed phase of the polymer in said system;
D) determining a concentration (c_{con}) of the condensed phase of the polymer and a concentration (c_{dil}) of the dilute phase of the polymer in said system by using a following linear form equation:
$$\frac{{V}_{c o n}}{{V}_{t o t}} = \frac{1}{{c}_{con} - {c}_{dil}} \cdot {c}_{tot} - \frac{{c}_{dil}}{{c}_{con} - {c}_{dil}} ,$$
D′) comparing the measured condensed volume (V_{con}), the measured condensed concentration (c_{con}), and/or the measured volume fraction (V_{con}/Vₜₒₜ) of said polymer in the system with a condensed volume (V_{ref}), a condensed concentration (c_{ref}), and/or a volume fraction (V_{ref}/Vₜₒₜ) of the polymer in the presence of a reference molecule in a control system;
F) identifying the bioactive compounds from the test compounds;
and a device comprises a plurality of systems.

12. The assay method according to claim 11, wherein the condensed volume (V_{ref}), the condensed concentration (c_{ref}), and/or the volume fraction (V_{ref}/Vₜₒₜ) of the polymer in the presence of a reference molecule is (are) predetermined or measured at the same time.

13. The assay method according to claim 11, wherein the identified bioactive compound(s) increase(s) the measured condensed volume (V_{con}), the measured condensed concentration (c_{con}), the measured dilute concentration (c_{dil}), and/or the measured volume fraction (V_{con}/Vₜₒₜ) of the polymer obtained by the step C) and/or D) than the predetermined condensed volume (V_{ref}), the measured condensed concentration (c_{ref}), and/or the measured volume fraction (V_{ref}/Vₜₒₜ) of the polymer of said polymer in the presence of a reference molecule in the control system.

14. A device for determining a binodal curve of a polymer in a system comprising:
i) means for measuring a total volume (Vₜₒₜ) of the system;
ii) means for measuring a volume fraction (V_{con}/Vₜₒₜ) of a condensed phase of the polymer in said system;
iii) means for calculating a concentration (c_{con}) of the condensed phase of the polymer and a concentration (c_{dil}) of the dilute phase of the polymer in said system by using a following linear form equation:
$$\frac{{V}_{c o n}}{{V}_{t o t}} = \frac{1}{{c}_{con} - {c}_{dil}} \cdot {c}_{tot} - \frac{{c}_{dil}}{{c}_{con} - {c}_{dil}} .$$

15. The device according to claim 14, wherein the means for calculating a concentration (c_{con}) of the condensed phase of the polymer and a concentration (c_{dil}) of the dilute phase of the polymer in said system is a computer which is connected to a controller and a detector, wherein the computer is configured to receive signals detected from the detector and wherein the computer is configured to calculate a concentration (c_{con}) of the condensed phase of the polymer and a concentration (c_{dil}) of the dilute phase of the polymer in said system by using a following linear form equation:
$$\frac{{V}_{c o n}}{{V}_{t o t}} = \frac{1}{{c}_{con} - {c}_{dil}} \cdot {c}_{tot} - \frac{{c}_{dil}}{{c}_{con} - {c}_{dil}} .$$

16. The method according to claim 1, wherein the polymer is a mixture of polymers, and the mixture of polymers is selected from a group comprising or consisting of a mixture of proteins, a mixture of RNAs, a mixture of DNAs, a mixture of proteins and RNAs, a mixture of proteins and DNAs, and a mixture of proteins, RNAs and DNAs.

17. The method according to claim 7, further comprising after the step C2a); c2b) adjusting the measurement fluorescent image with a predetermined partition factor.


SEQUENCE LISTING (3 sequences)

SEQ ID NO: 1 - protein, 797 aa, synthetic construct
  REGION 1..797: note = GFP-tagged Fused in Sarcoma protein purified from BAC construct
MASNDYTQQATQSYGAYPTQPGQGYSQQSSQPYGQQSYSGYSQSTDTSGYGQSSYSSYGQSQNTGYGTQSTPQGYGSTGGYGSSQSSQSSYGQQSSYPGYGQQPAPSSTSGSYGSSSQSSSYGQPQSGSYSQQPSYGGQQQSYGQQQSYNPPQGYGQQNQYNSSSGGGGGGGGGGNYGQDQSSMSSGGGSGGGYGNQDQSGGGGSGGYGQQDRGGRGRGGSGGGGGGGGGGYNRSSGGYEPRGRGGGRGGRGGMGGSDRGGFNKFGGPRDQGSRHDSEQDNSDNNTIFVQGLGENVTIESVADYFKQIGIIKTNKKTGQPMINLYTDRETGKLKGEATVSFDDPPSAKAAIDWFDGKEFSGNPIKVSFATRRADFNRGGGNGRGGRGRGGPMGRGGYGGGGSGGGGRGGFPSGGGGGGGQQRAGDWKCPNPTCENMNFSWRNECNQCKAPKPDGPGGGPGGSHMGGNYGDDRRGGRGGYDRGGYRGRGGDRGGFRGGRGGGDRGGFGPGKMDSRGEHRQDRRERPYGAPGSAGSAAGSGMVSKGEELFTGVVPILVELDGDVNGHKFSVSGEGEGDATYGKLTLKFICTTGKLPVPWPTLVTTLTYGVQCFSRYPDHMKQHDFFKSAMPEGYVQERTIFFKDDGNYKTRAEVKFEGDTLVNRIELKGIDFKEDGNILGHKLEYNYNSHNVYIMADKQKNGIKVNFKIRHNIEDGSVQLADHYQQNTPIGDGPVLLPDNHYLSTQSKLSKDPNEKRDHMVLLEFVTAAGITLGMDELYKLEVLFQGPGSSHHHHHHSG

SEQ ID NO: 2 - protein, 953 aa, synthetic construct
  REGION 1..953: note = PGL-3-6xHis-mEGFP protein purified in SF9 insect cells from baculovirus construct
MEANKRQIVEVDGIKSYFFPHLAHYLASNDELLVNNIAQANKLAAFVLGATDKRPSNEEIAEMILPNDSSAYVLAAGMDVCLILGDDFRPKFDSGAEKLSQLGQAHDLAPIIDDEKKISMLARKTKLKKSNDAKILQVLLKVLGAEEAEEKFVELSELSSALDLDFDVYVLAKLLGFASEELQEEIEIIRDNVTDAFEACKPLLKKLMIEGPKIDSVDPFTQLLLTPQEESIEKAVSHIVARFEEASAVEDDESLVLKSQLGYQLIFLVVRSLADGKRDASRTIQSLMPSSVRAEVFPGLQRSVFKSAVFLASHIIQVFLGSMKSFEDWAFVGLAEDLESTWRRRAIAELLKKFRISVLEQCFSQPIPLLPQSELNNETVIENVNNALQFALWITEFYGSESEKKSLNQLQFLSPKSKNLLVDSFKKFAQGLDSKDHVNRIIESLEKSSSSEPSATAKQTTTSNGPTTVSTAAQVVTVEKMPFSRQTIPCEGTDLANVLNSAKIIGESVTVAAHDVIPEKLNAEKNDNTPSTASPVQFSSDGWDSPTKSVALPPKISTLEEEQEEDTTITKVSPQPQERTGTAWGSGDATPVPLATPVNEYKVSGFGAAPVASGFGQFASSNGTSGRGSYGGGRGGDRGGRGAYGGDRGRGGSGDGSRGYRGGDRGGRGSYGEGSRGYQGGRAGFFGGSRGGSSRENLYFQSSAHHHHHHHVMVSKGEELFTGVVPILVELDGDVNGHKFSVSGEGEGDATYGKLTLKFICTTGKLPVPWPTLVTTLTYGVQCFSRYPDHMKQHDFFKSAMPEGYVQERTIFFKDDGNYKTRAEVKFEGDTLVNRIELKGIDFKEDGNILGHKLEYNYNSHNVYIMADKQKNGIKVNFKIRHNIEDGSVQLADHYQQNTPIGDGPVLLPDNHYLSTQSALSKDPNEKRDHMVLKEFVTAAGITLGMDELYKGA

SEQ ID NO: 3 - RNA, 250 nt, synthetic construct
  misc_feature 1..250: note = Poly(A) RNA
  source 1..250: mol_type = other RNA
aaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa